(19) United States
(12) Patent Application Publication
(10) Pub. No.: US 2009/0162896 A1
(43) Pub. Date: Jun. 25, 2009

(54) Production of Recombinant Collagen Like Proteins

(76) Inventor: Thomas Scheibel, Bayreuth (DE)

Correspondence Address: JENKINS, WILSON, TAYLOR & HUNT, P. A., Suite 1200 UNIVERSITY TOWER, 3100 TOWER BLVD.,, DURHAM, NC, 27707, US

(21) Appl. No.: 12/085,266

(22) PCT Filed: Nov. 17, 2006
(86) PCT No.: PCT/EP2006/011061

§ 371 Date: Jan. 21, 2009

(30) Foreign Application Priority Data
Nov. 17, 2005 (EP) 05025131.3

(51) Int. Cl. C12P 21/00; C12N 1/19; C07K 14/435; C12N 15/63

(52) U.S. Cl. 435/69.1; 435/254.2; 435/254.21; 435/254.22; 435/254.23; 435/320.1; 530/350

(57) ABSTRACT

The present invention is directed to a yeast cell for producing a recombinant collagen like protein. The present invention is further directed to a kit of parts or a co-expression system for use in the production of such a protein and to a method of producing said recombinant protein and a thread made therefrom. Furthermore, the invention pertains to proteins or threads obtainable by these methods as well as their use in various fields of technology and medicine.

The present invention is directed to a yeast cell for producing a recombinant collagen like protein. The present invention is further directed to a kit of parts or a co-expression system for use in the production of such a protein and to a method of producing said recombinant protein and a thread made therefrom. Furthermore, the invention pertains to proteins or threads obtainable by these methods as well as their use in various fields of technology and medicine.

Marine mussels are found in the turbulent habitat of the inter-tidal zone and here, marine mussels have been very successful in colonizing rocks, which are exposed to wind and waves. This success is partially due to a unique anchorage by which they fix themselves on the solid surfaces of the rocks. A part of this anchorage is a fibrilar structure, known as “byssus” or also known as “mussel silk”. The byssus provides mussels with the necessary tenacity to survive the incessant buffeting of waves by attaching to rocks or hard surfaces.

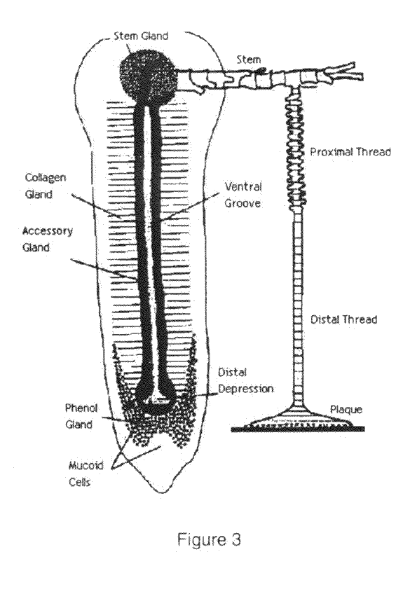

The mussel byssus is completely consisting of extra-cellular matrix which is forming a bundle of short threads that resemble tiny tendons [2]. Byssus threads show unusual mechanical properties, since they resemble soft rubber at one end and rigid nylon at the other and these properties are found with a seamless and gradual transition [4]. Byssal threads are also elastomeric: they are able to withstand significant deformations without rupture and can return to their original state, when the stress is removed [5]. At the distal end, the byssus threads are fixed by adhesive plaques at the rock. At the proximal ends, the byssus threads are combined to a so-called byssus stem, which is anchored at the base of the mussel foot (see FIG. 3).

The byssus threads of marine mussels are elastomeric fibers with a great capacity of absorbing and dissipating energy. Up to 70% of the total absorbed energy can be dissipated in the byssus. In Mytilus species (M. edulis and M. galoprovincialis), each new thread has dimensions of a few centimeters in length and less than 0.1 cm in diameter and is produced in ca. 5 minutes in the ventral groove of the foot by a process akin to reaction injection molding [3].

Morphologically, the byssus is divided into four sections (from proximal to distal): root, stem, thread and plaque or pad. Furthermore, the thread is further subdivided into proximal and distal portions according to appearance, i.e. smooth and stiff for the distal, soft and weaker for the proximal portion.

Byssus threads are elastomeric. The Young's modulus is low (in the range of from 10-500 MPa), the extensibility can be as high as 200% and there is restorative recall. In common with other protein elastomers as elastin, resiline and abductine, byssus threads are quite tough. Thoughness and energy dissipation are both crucial properties for holdfasts. Energy dissipation in fibers subjected to cyclic stress-strain-analysis is frequently normalized with respect to the total absorbed energy and reported as hysteresis or percentage hysteresis.

The stress-strain cycle for one thread has been dissected into separate mechanical contributions for the distal and proximal portions of the thread. As mentioned above, of these, the distal portion is stronger, stiffer and superior at damping whereas the proximal portion is softer and weaker with a lower, but still significant hysteresis.

The mechanical properties of byssus threads are further complicated by time- and strain-dependent behavior. It was demonstrated that, when strained beyond its yield point, the distal portion exhibited a schematic stress softening, i.e. the initial modulus of the second cycle was reduced to about 20% of the modulus in the first cycle (500-80 MPa). The complete recovery of the modulus of the first cycle was slow, e.g. longer than 24 h but significant partial recovery can occur within 1 h (30% of the original values). The proximal portion also shows a tendency to change stiffness with cyclic loading. In this case, there is strain-stiffening from an initial modulus of 35 MPa to an asymptotic leveling at 50 MPa, an increase of about 40%.

MASCOLO and WAITE (1986) first identified chemical gradients in byssus threads in Mytilus. After treatment of the threads with pepsin, two pepsin-resistant collagen fragments, called ColP and ColD, having molecular weights of 50 kDa and 60 kDa, respectively were identified. ColP can be found predominantly in the proximal area and is hardly to be found in the distal area. In contrast, the amount of ColD increases in the distal part to approximately 100% (LUCAS et al., 2002; QIN & WAITE, 1995). In the byssus thread as well as in the mussel foot, there is a further collagen-like protein which takes part in the construction of the thread structure. This additional protein is called ColNG (NG=no gradient), and is, in contrast to ColD and ColP, evenly distributed throughout the whole thread. Its physiological function presumably is being an adapter between the two other thread collagens (QIN & WAITE, 1998).

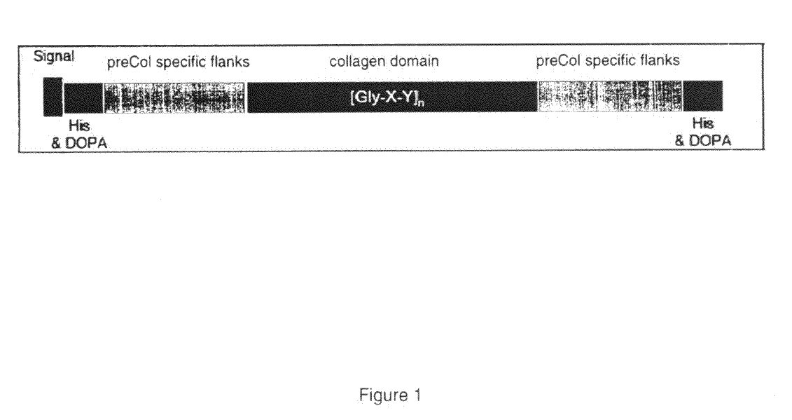

The Pepsin-cleaved fragments ColD and P originate from the so-called preCollagens P and D. Both preCol's (i.e. D and P) from M. edulis are characterized by a common basic structure: a central collagen helix which is flanked by different flanking regions, which are each terminated by a histidine and DOPA rich terminus (see FIG. 1).

The mechanism for the assembly of byssus collagens into fibers has been an elusive aspect of the byssus biochemistry. It is well recognized that the collagens undergo stabilization via cross-linking; however the chemistry is still not well understood. There are two distinct cross-linking possibilities: metal complexation and covalent bond formation between collagen units [8, 9]. Metal complexation is suggested by the high levels of iron, copper, nickel and zinc found in byssus and by the occurrence of metal-binding histidine-rich sequences in both terminals of the byssal proteins. Moreover, DOPA is present in both the termini of all Pre-Col's. Peptidyl-DOPA provides excellent metal binding sites and peptidyl-DOPA-Fe(III) chelates have been reported in the marine adhesive plaque mefp-1 [10]. Further, it has been shown that removal of metal ions from byssal fiber by EDTA reduces the yield strength of the fiber. Covalent cross-links have also been observed. They are generally formed by oxidative coupling between tyrosines, DOPA and cysteines. In a study of byssus stressed by conditions of high flow and aeration, the primary product of oxidation was found to be 5,5′-diDOPA[11]. Other possible coupling products like the Michael-type addition of lysines to oxidized DOPA have not been found [7].

Like “normal” collagen, each mussel collagen has a signal sequence of 20 amino acids which make sure that the alpha-chains are transported into the endoplasmatic reticulum. There, three identical alpha-chains assemble to a homotrimer. The ColD alpha-chain, which means the pepsin-cleaved preColD, has a molecular mass of 60 kDa by SDS-PAGE and 47 kDa by MALDI-TOF mass spectometry (QIN et al., 1997). The alpha-chain of ColP, which means the pepsin-cleaved preColP, has a molecular mass of 55 kDa (by SDS-PAGE) and 40 kDa (MALDI-TOF), respectively (COYNE et al., 1997). The precursors of the alpha-chain are named preColD and preColP and have molecular masses of 95 and 97 kDa by means of SDS-PAGE analysis and 75 and 80 kDa respectively by analysis with MALDI-TOF mass spectometry (COYNE et al., 1997; QIN et al., 1997). Both collagens have characteristics which are typical for collagen type I-III. Both have an amount of more than 34% of glycine and show a proline and hydroxyproline content of combined 20% within the collagen domain.

The flanking regions fully correspond to other structural proteins, namely elastin (preColP) and silk-fibroin (preColD). This structural construction gives an explanation for the mechanical behavior of mussel byssi. For this reason, it would be highly relevant to recombinantly produce the underlying mussel byssus collagens in order to use these extraordinary natural materials as building blocks in new technological applications.

The development of materials having defined characteristics, in particular of materials which are capable of regenerate themselves following stress or overloading has been of high interest in the material sciences for a long time. Composite structures are of gaining interest in technology, in particular for electronic components and devices, energy converters and other materials. By combination of materials having different mechanical characteristics, structural interfaces will be formed causing new technological problems.

Thus, for many applications it would highly desirable to provide a graduated structure thereby reducing the overall load of the material.

Furthermore, the use and application of mussel collagens in medicine is of great interest because of the high potential biocompatibility. Based on this, medical transplants and tissues could be generated having a high degree of immunocompatibility. The production of recombinant mussel collagens is an interesting and important technical problem which has to be solved before technical applications of mussel collagens may be envisioned.

Therefore it is an object underlying the present invention to provide recombinant mussel byssus proteins having enhanced characteristics as, in particular, improved capability of being expressed in high yield and good strength and flexibility. It is a further object of the present invention to provide recombinant mussel byssus proteins which can be specifically adapted to the required application by specific arrangement of the building blocks on which they are based to provide a graduated structure. Furthermore, it is an object of the present invention to provide expression vectors coding for recombinant mussel byssus proteins, which can be conveniently expressed in already known eucaryotic expression systems. Additionally, it is an object of the present invention to provide improved paper, textile and leather products. Additional objects are to provide new proteins and further materials based on recombinant mussel byssus proteins such as spheres, nanofibrils, hydrogels, threads, foams, films for use in biotechnology, medicine, pharmaceutical and food applications, cosmetics, in electronic devices and for other commercial purposes. It is a still further object of the present invention to provide a host cell, which is capable of expressing collagen like proteins, in particular mussel byssus proteins, in high yield and quality.

These objects are solved by the subject-matter of the independent claims. Preferred embodiments are set forth in the dependent claims.

Up to now, the expression of recombinant mussel byssus proteins has never been shown. This might be at least partially due to the complex process of expressing those proteins and threads made therefrom. The complexity of the biosynthesis of collagen leads to a reduced predictability of the outcome of any attempt to express recombinant collagens and, therefore, these attempts might presumably lead to improperly folded proteins, low yield or, in the worst case, to no expression of collagen at all.

In the present invention, a host cell system is provided which results in high yields of properly folded collagen like proteins, in particular of mussel byssus proteins.

The present invention in particular is directed to the following aspects and embodiments:

According to a first aspect, the present invention provides a yeast cell for producing a recombinant collagen like protein, in particular mussel byssus protein, which yeast cell has been transformed with the following elements:

a) a first expression vector which codes for said recombinant collagen like protein; and
b) a second expression vector comprising a nucleic acid coding for prolyl-4-hydroxylase (P4H).

Due to the complexity of the biosynthesis of collagen, for the recombinant synthesis of collagen-like proteins, the inventors found out that some factors have to be considered, the most important one being the posttranslational modification in the endoplasmatic reticulum (ER) of proline to hydroxyproline by prolyl-4 hydroxylase, a tetrameric enzyme, which is composed of the two sub-units of alpha-PH (=P4HA) and PDI (=P4HB) (BULLEID et al., 2000). For this reason, procaryotic expression systems, for example bacterial expression systems, may not be used in the present invention.

Yeasts on the one hand offer the cell compartmentation which is required for the synthesis of collagen, on the other hand, however, they are lacking the enzyme prolyl-hydroxylase (P4H) which is required for the synthesis of collagen. Apart therefrom, yeasts would be a desirable expression system for recombinant collagens since their cultivation, also in large scale expression systems, is comparably easy to achieve and the yield of recombinant protein therefrom is superior to other expression systems. Thus, expression in yeast might lead to an efficient (and also cost-effective) production of recombinant collagen like proteins, in particular of mussel byssus proteins. However, as a result of the above drawbacks of yeast cells, an expression of those proteins in yeast cells has not been achieved up to now.

It could be shown by the inventors for yeast cells, which do not possess P4H, that human P4H subunits can be produced recombinantly and can be correctly folded. Apart therefrom, it could be shown for these yeast strains that by co-expression of both human P4H subunits, the synthesis of mussel byssus collagen is possible and folded, stabile collagen is formed. Interestingly, the co-expression of the genes of both P4H subunits is sufficient for the formation of a stable triple helix in yeast and no further enzymes or folding promoters or chaperones specific for collagen are required, as for example Hsp47, or in other words, the chaperones which are inherent to yeast are sufficiently “active”. Human collagens, recombinantly produced in yeast possessed the same content of hydroxyproline and, furthermore, are identical in respect to many other characteristics compared to native collagens.

By efficient transport in the ER of yeast, signal sequences of the co-expressed P4H subunits play an important role. A maximum sufficiency of localization can be achieved by replacing in a preferred embodiment, the human with a yeast signal sequence, for example from the S. cerevisae pheromone mating factor alpha1 (MFa). The P4H subunits modified by the MFa signal sequence were effectively transported into the lumen of the ER.

More preferably, the signal sequence is mating factor alpha 1 (MFa) of S. cerevisiae according to SEQ ID NO: 10.

As a yeast cell, preferably S. cerevisiae, Schizosaccharomyces pombe, Pichia pastoris, Candida albicans, or Hansenula polymorpha cells might be used.

The first expression vector preferably further comprises one or more regulatory elements. The expression vector must be suitable for expression in yeast cells.

Preferably, the regulatory elements contain a promoter selected from constitutive or inducible promoters, more specifically from GPD, GAL4, CUP1, MET25, GAL1 or GAL1-10.

In a further embodiment, the expression vector is a plasmid.

Preferably, the recombinant collagen like protein is a recombinant mussel byssus protein comprising or consisting of one or more fragments of a collagen domain flanked by elastin or silk fibroin.

This recombinant mussel byssus protein is composed of one or more types of building blocks, which provide different characteristics to the protein formed: as mentioned above, elastin and silk-fibroin have certain mechanical characteristics, which can give an explanation for the mechanical behavior of mussel byssi and, thus, also for the design of recombinantly produced mussel byssus proteins.

Therefore, these fragments can be used as one single type of fragment only, or, as an alternative, the recombinant protein can comprise two or more different fragments. For example, if great elasticity is wanted, the protein may only or predominantly comprise fragments of collagen flanked by elastin. If great stiffness and strength is required, the protein may comprise fragments of collagen flanked by silk-fibroin. As a further and preferred alternative, the protein may comprise a mixture of both types of fragments, for example forming a gradient from one region to the other. Thus, a protein/thread can be formed having specifically adapted configurations, i.e. parts having higher elasticity and parts having higher stiffness etc.

The term “flanked” means that elastin (or silk-fibroin) is present on both sides of the collagen domain.

The above fragments may be naturally derived, for example, the fragments may be obtained from Mytilus sp., preferably from M. edulis, M. galloprovincialis, M. californians, or Geukeria demissa.

According to a preferred embodiment, the recombinant mussel byssus protein of the invention comprises or consists of one or more of the fragments preColP and/or preColD or variants thereof. These fragments have been outlined above. Both preCol's (i.e. D and P) are derived from M. edulis and are characterized by a common basic structure: a central collagen helix which is flanked by different flanking regions, which are each terminated by a histidine and DOPA rich terminus (see FIG. 1). The flanking regions fully correspond to known structural proteins, namely elastin (preColP) and silk-fibroin (preColD).

The sequences of preColP and preColD are translated from the respective nucleic acids. Therefore, whenever amino acids are recited herein in the following, they are referring to preColP and preColD and these sequences will be used in the various technological applications mentioned hereinabove. The nucleic acid sequences mentioned herein in the first place are directed to preColP and preColD encoding sequences.

According to a further embodiment, the recombinant protein of the invention comprises or consists of one or more fragments of SEQ ID NO: 3 and/or 4 or variants thereof. The Seq ID No's reflect the sequences of preColP and preColD.

As mentioned above, the present invention also comprises variants of those amino acid sequences. For example, said variants may contain one or more substitutions, insertions and/or deletions when compared to the amino acid sequences mentioned above.

In particular variants of the protein, for example deletions, insertions and/or substitutions in the sequence, which cause so-called “silent” changes, are considered to be part of the invention.

Preferably are such amino acid substitutions the result of substitutions which substitute one amino acid with a similar amino acid with similar structural and/or chemical properties, i.e. conservative amino acid substitutions.

Amino acid substitutions can be performed on the basis of similarity in polarity, charges, solubility, hydrophobic, hydrophilic, and/or amphipathic (amphiphil) nature of the involved residues. Examples for hydrophobic amino acids are alanine, leucine, isoleucine, valine, proline, phenylalanine, tryptophan and methionine. Polar, neutral amino acids include glycine, serine, threonine, cysteine, thyrosine, asparagine and glutamine. Positively (basic) charged amino acids include arginine, lysine and histidine. And negatively charged amino acids include aspartic acid and glutamic acid.

“Insertions” or “deletions” usually range from one to five amino acids. The allowed degree of variation can be experimentally determined via methodically applied insertions, deletions or substitutions of amino acids in a polypeptide molecule using recombinant DNA methods. The resulting variants can be tested for their characteristics, in particular their mechanical characteristics.

It is noted that the term “variant” as used herein also comprises the above amino acid sequences of preColP and preColD, wherein the first 19 amino acids constituting the original mussel signal sequence were replaced by other signal sequences. A preferred example hereof is replacement of the mussel signal sequence by signal sequence alpha MF (SEQ ID NO: 10: “MRFPSIFTAV LFAASSALA”). This signal sequence in particular is suitable for expression of the nucleic acids in yeasts.

The present invention also provides an isolated nucleic acid encoding the recombinant protein as defined above. The term “isolated” as used herein with reference to nucleic acids refers to a naturally-occurring nucleic acid that is not immediately contiguous with both of the sequences with which it is immediately contiguous (one on the 5′ end and one on the 3′ end) in the naturally-occurring genome of the organism from which it is derived.

For example, an isolated nucleic acid can be, without limitation, a recombinant DNA molecule of any length, provided one of the nucleic acid sequences normally found immediately flanking that recombinant DNA molecule in a naturally-occurring genome is removed or absent. Thus, an isolated nucleic acid includes, without limitation, a recombinant DNA that exists as a separate molecule (e.g., a cDNA or a genomic DNA fragment produced by PCR or restriction endonuclease treatment) independent of other sequences as well as recombinant DNA that is incorporated into a vector, an autonomously replicating plasmid, a virus (e.g., a retrovirus, adenovirus, or herpes virus), or into the genomic DNA of a prokaryote or eukaryote. In addition, an isolated nucleic acid can include a recombinant DNA molecule that is part of a hybrid or fusion nucleic acid sequence.

The term “isolated” also includes any non-naturally-occurring nucleic acid since non-naturally-occurring nucleic acid sequences are not found in nature and do not have immediately contiguous sequences in a naturally-occurring genome. For example, non-naturally-occurring nucleic acid such as an engineered nucleic acid is considered to be isolated nucleic acid. Engineered nucleic acid can be made using common molecular cloning or chemical nucleic acid synthesis techniques. Isolated non-naturally-occurring nucleic acids can be independent of other sequences, or incorporated into a vector, an autonomously replicating plasmid, a virus (e.g., a retrovirus, adenovirus, or herpes virus), or the genomic DNA of a prokaryote or eukaryote. In addition, a non-naturally-occurring nucleic acid can include a nucleic acid molecule that is part of a hybrid or fusion nucleic acid sequence.

It will be apparent to those of skill in the art that a nucleic acid existing among hundreds to millions of other nucleic acid molecules within, for example, cDNA or genomic libraries, or gel slices containing a genomic DNA restriction digest is not to be considered an isolated nucleic acid.

A nucleic acid encoding the above amino acids may be a nucleic acid sequence coding for the mature or the immature amino acid sequence of the recombinant mussel byssus protein.

In a preferred embodiment, the isolated nucleic acid comprises or consists of the nucleic acid of SEQ ID NO: 1 and/or 2 or variants thereof. These variants are each defined as having one or more substitutions, insertions and/or deletions as compared to the sequences of SEQ ID NO: 1 or 2, provided that said variants hybridize under moderately stringent or stringent conditions to a nucleic acid which comprises the sequence of SEQ ID NO: 1 or 2, or provided that said variants comprise nucleic acid changes due to the degeneracy of the genetic code, which code for the same or a functionally equivalent amino acid as the nucleic acid sequence of SEQ ID NO: 1 or 2.

As mentioned above, the present invention also encompasses a variant of said nucleic acids,

wherein the nucleic acids coding for the first 19 amino acids (signal sequence) were replaced, preferably by the yeast signal sequence MFa (SEQ ID NO: 10).

Stringency of hybridization, as used herein, refers to conditions under which polynucleotide duplexes are stable. As known to those of skill in the art, the stability of duplex is a function of sodium ion concentration and temperature (see, for example, Sambrook et al., Molecular Cloning: A Laboratory Manual 2^{nd} Ed. (Cold Spring Harbor Laboratory, (1989)). Stringency levels used to hybridize can be readily varied by those of skill in the art.

Stringent washing conditions mean 0.2×SSC (0.03 M NaCl, 0.003 M sodium citrate, pH 7)/0.1% SDS at 65° C. For shorter fragments, e.g. oligonucleotides up to 30 nucleotides, the hybridization temperature is below 65° C., for example at 50° C., preferably above 55° C., but below 65° C. Stringent hybridization temperatures are dependent on the size or length, respectively of the nucleic acid and their nucleic acid composition and will be experimentally determined by the skilled artisan. Moderate stringent hybridization temperatures are for example 42° C. and washing conditions with 0.2×SSC/0.1% SDS at 42° C.

The P4H used in the present invention preferably is human or mussel P4H.

In a second aspect, a kit of parts or a co-expression system comprising the following constituents is provided:
a) the first expression vector as defined herein; and
b) the second expression vector as defined above.

This kit of parts or co-expression system may be efficiently used in expressing the recombinant mussel byssus protein in yeast cells.

In a still further aspect, a method of producing recombinant collagen like proteins, in particular mussel byssus proteins is disclosed comprising the steps of:
a) providing a yeast cell as defined hereinabove;
b) transforming said yeast cell with an expression vector or the co-expression system explained above;
c) expressing recombinant protein from said host cell under suitable conditions; and
d) recovering said protein.

Furthermore, a method for producing threads from recombinant mussel byssus protein is provided, comprising the following steps:
a) providing recombinant protein produced in accordance with the above method, and
b) spinning or moulding said protein into threads by a suitable method.

The spinning may preferably be done by electrospinning. Electrospinning is a fiber formation technique that uses electrostatic forces to create continuous, nanometer diameter fibers. A wide variety of natural and artificial polymers have been electrospun from the solution and melt phase and are of interest for an assortment of application areas that require high surface area materials (filtration membranes and biomedical devices).

An additional aspect of the invention is a protein or thread obtainable by one of the above methods.

The proteins/threads of the invention find application preferably in the field of biotechnology and/or medicine.

For example, they might be used for the manufacture of wound closure or coverage systems or suture materials. Furthermore, the proteins/threads may preferably be used for the manufacture of replacement materials, preferably artificial cartilage or tendon materials.

Additionally, the threads/proteins of the invention can be used in the manufacture of medical devices such as medical adhesive strips, skin grafts, replacement ligaments, and surgical mesh; and in a wide range of industrial and commercial products, such as clothing fabric, bullet-proof vest lining, container fabric, bag or purse straps, cable, rope, adhesive binding material, non-adhesive binding material, strapping material, automotive covers and parts, aircraft construction material, weatherproofing material, flexible partition material, sports equipment; and, in fact, in nearly any use of fiber or fabric for which high tensile strength and elasticity are desired characteristics. Adaptability and use of the stable fiber product in other forms, such as a dry spray coating, bead-like particles, or use in a mixture with other compositions is also contemplated by the present invention.

It is explicitely noted that preferred applications of the mussel byssus collagens of the present invention are in the manufacture and processing of clothing fabric (textiles) and leather, automotive covers and parts, aircraft construction materials as well as in the manufacture and processing of paper.

The recombinant mussel byssus proteins of the present invention may be added to cellulose and keratin and collagen products and thus, the present invention is also directed to a paper or a skin care and hair care product, comprising cellulose and/or keratin and/or collagen and the proteins of the present invention. Papers and skin care and hair care products, in which the proteins of the present invention are incorporated are showing improved characteristics, in particular improved tensile strength or tear strength.

Furthermore, the recombinant mussel byssus proteins of the invention may be used as a coating for textile and leather products, thereby conferring stability and durability to the coated product. The proteins in particular show applicability for coating leather products, since in this case, tanning and its negative effects for environment can be avoided or at least reduced.

The invention is also directed to products containing said mussel byssus proteins, for example, wound closure or coverage systems, suture materials, replacement materials, preferably artificial cartilage or tendon materials, cosmetics, drug delivery vehicles, fabrics, textile, paper product, leather product, automotive parts or aircraft parts. In general, it is also directed to materials based on recombinant mussel byssus proteins such as spheres, nanofibrils, hydrogels, foams, films.

Unless otherwise defined, all technical and scientific terms used herein have the same meaning as commonly understood by one of ordinary skill in the art to which this invention pertains. All publications, patent applications, patents, and other references mentioned herein are incorporated by reference in their entirety. In case of conflict, the present specification, including definitions, will control. In addition, the examples are illustrative only and not intended to be limiting.

The invention is now further illustrated by examples and the accompanying drawings, which are showing the following:

FIG. 1 is illustrating the general structure of mussel byssus collagens;

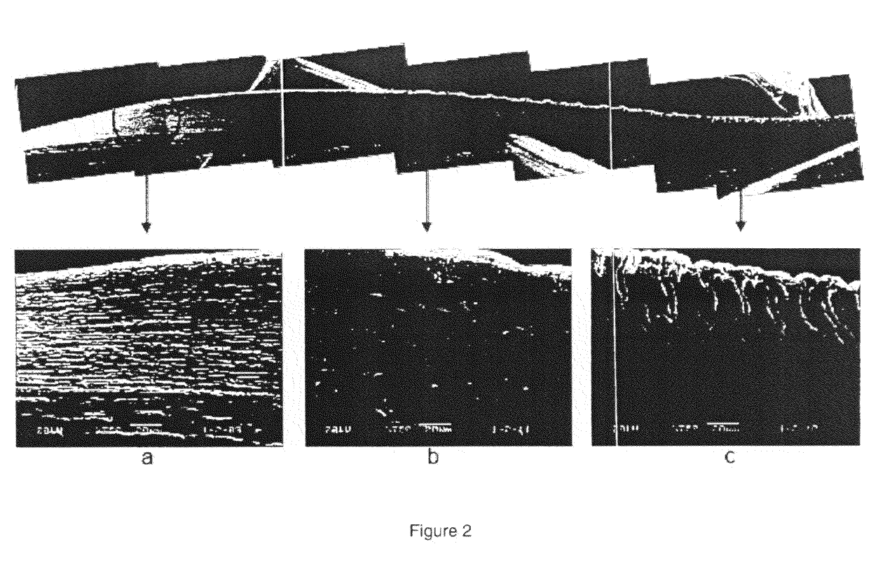

FIG. 2 depicts a series of SEM images of byssus threads in direction distal to proximal—the marked portions are each enlarged below. a) distal; b) median; c) proximal;

FIG. 3 shows the structure of mussel byssus;

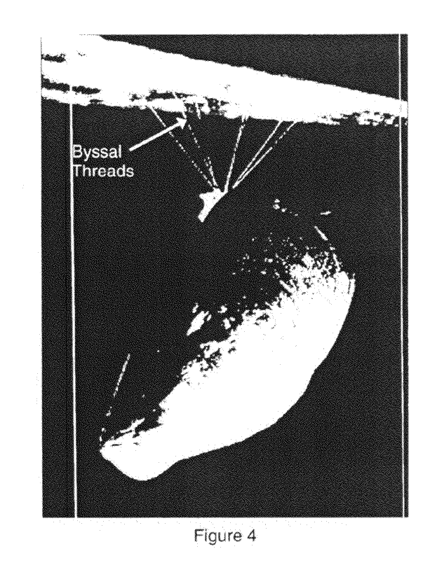

FIG. 4 illustrates a mussel adhered to a solid surface by byssus threads;

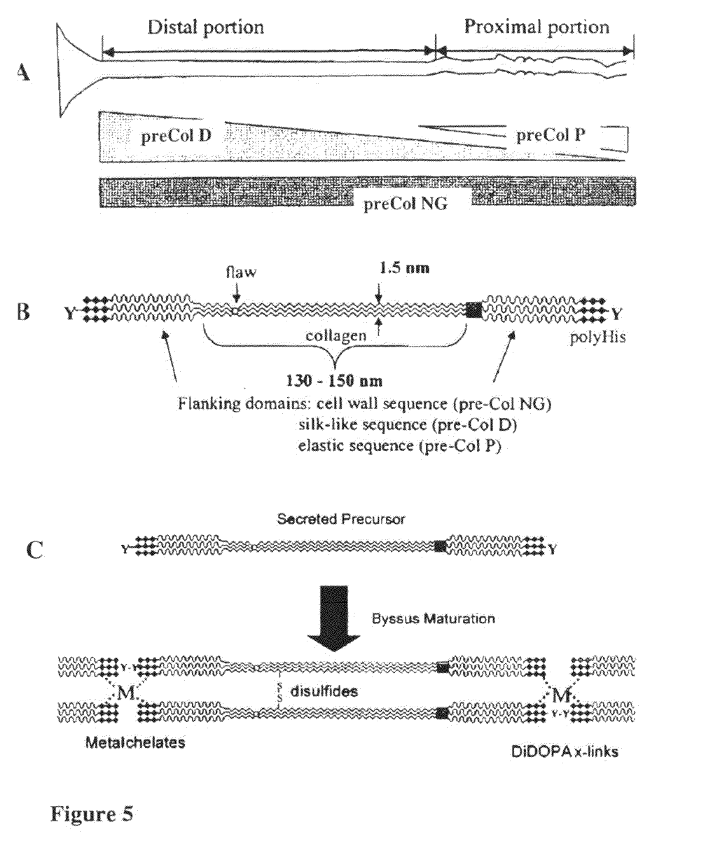

FIG. 5: (A) Distribution of preCols in the thread. (B) Schematic of a collagenous subunit with flanking domains. Terminal regions denoted by diamonds are His-rich. DOPA is designated by Y. (C) Model of cross-linking interactions between axial and lateral preCols;

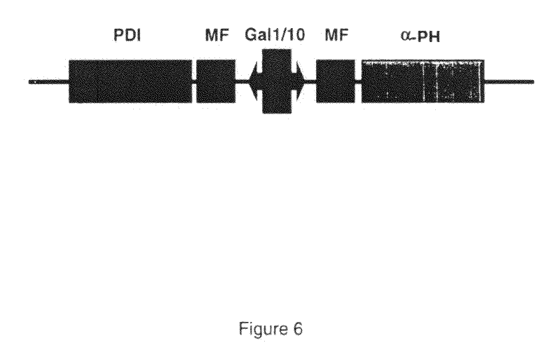

FIG. 6: Design of the P4H construct;

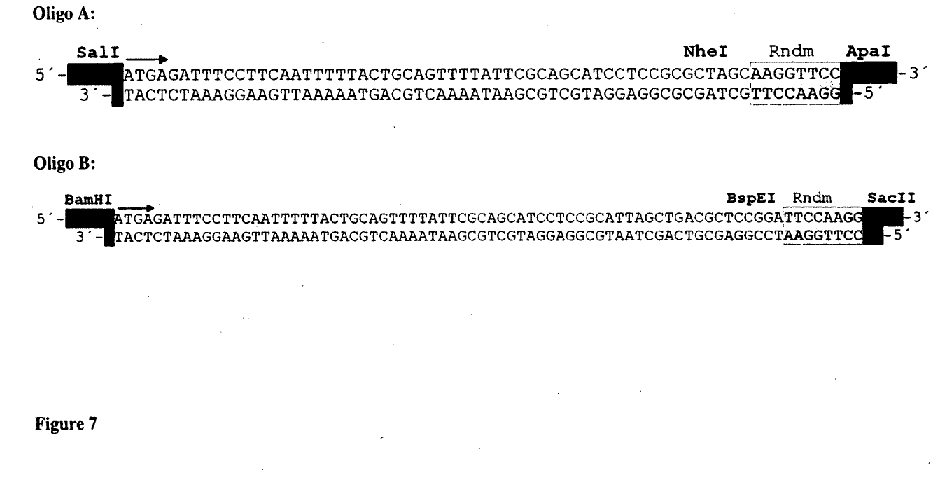

FIG. 7: Design of oligonucleotides to generate α-MF signal sequences ready to be cloned into respective expression plasmids;

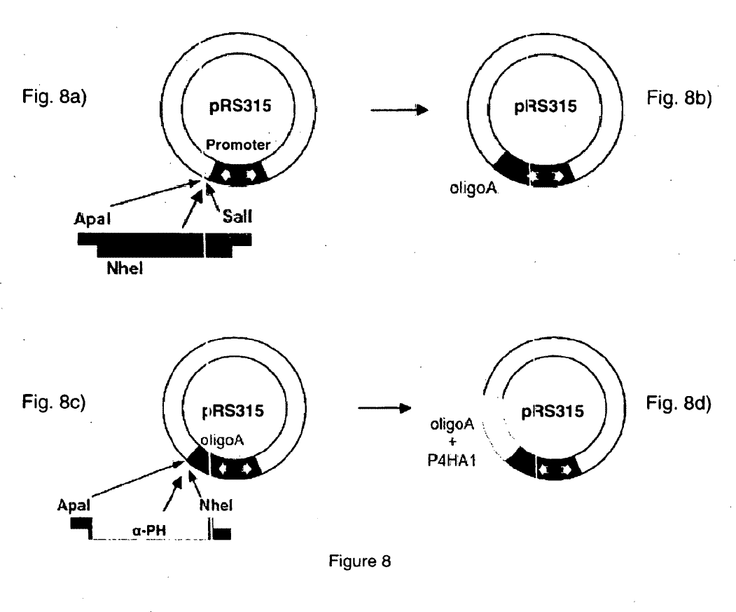

FIG. 8: Cloning strategy for α-PH;

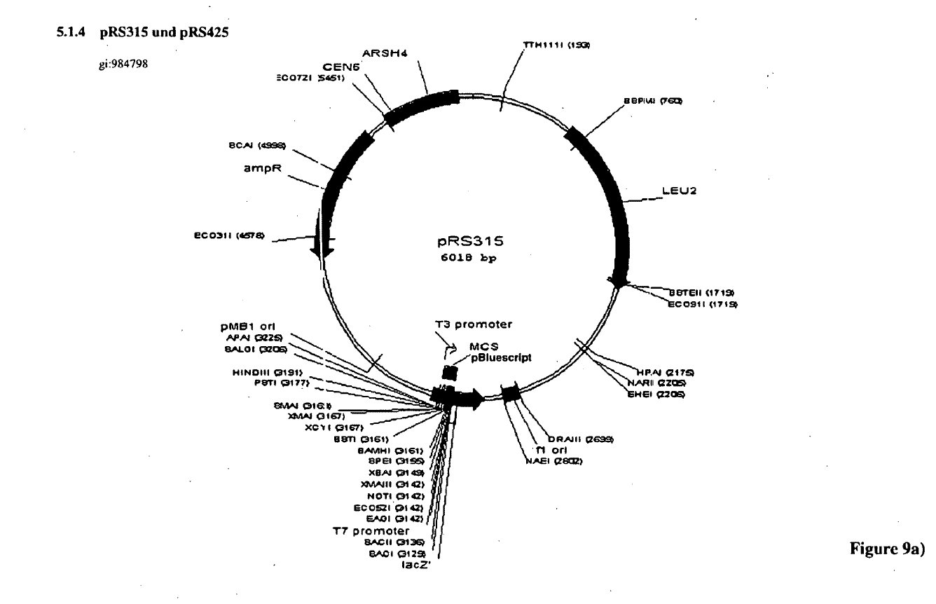

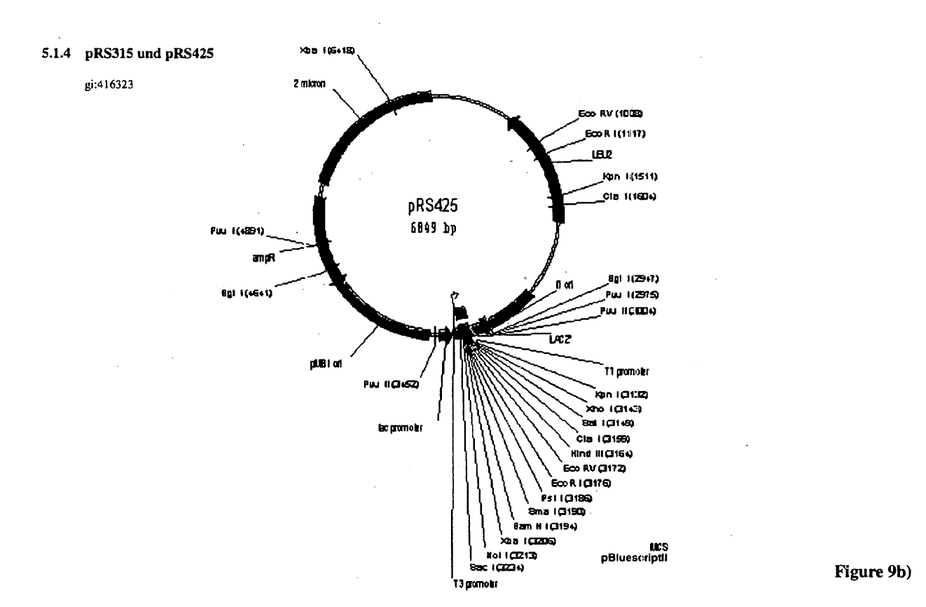

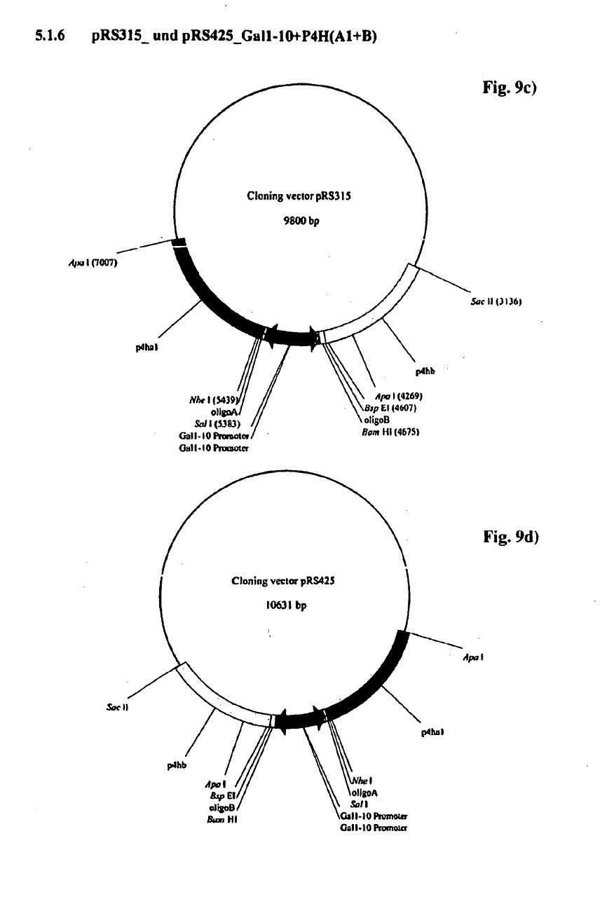

FIG. 9: Vector maps.

### EXAMPLES

### Expression of Collagen Proteins of Mussel Byssus in Yeast

Collagen synthesis in general reflects a complex biochemical process. The process requires e.g. post-translational modification of certain prolines of the respective collagens to 4-hydroxyproline in the ER by the enzyme Prolyl 4-hydroxylase (P4H). P4H, an α₂β₂ tetramer in vertebrates, plays a central role in the synthesis of collagens. 4-hydroxyproline residues, generated by P4H, are essential for the folding of the newly synthesized collagen polypeptide chains into triple-helical collagen molecules [13].

### Human Prolyl-4-Hydroxylase Expression Construct

The construct of P4H requires the cloning of a signal sequence into the yeast vector adjacent to the genes for the two subunits of P4H, α-PH and PDI. Both genes are placed under the control of a bi-directional promotor, which is induced in the presence of Galactose (Gal1/10) (see FIG. 6). The signal sequence is required for translocation of P4H subunits into the ER, where they can assemble into the native tetramer. Maximum efficiency for localization has been achieved when the human signal sequence is replaced by yeast's own signal sequence of the mating factor α-MF [12]. See FIG. 6 in this context.

The gene for α-PH (without signal sequence) is amplified by PCR from a c-DNA library from HepG2 liver cells (provided by Professor Adamski, GSF Munich, Germany), while the c-DNA of the beta-subunit (PDI) (without signal sequence) will be amplified from an E. coli cloning vector (provided by Professor Neil Bulleid, University of Manchester, UK). For each gene a respective αMF signal sequence will be engineered based on two single stranded oligonucleotides. The oligos A and B are planned in a way (see FIG. 7) that after annealing the double stranded DNA can be directly cloned into respective vectors.

The cloning strategy for α-PH is shown as an example (FIG. 8). Cloning of the cDNA of PDI will be performed in an identical way. Two different yeast vectors will be used: pRS315 (CEN, reflecting a single copy number plasmid) and pRS425 (2μ, reflecting a multi copy plasmid), both containing the bi-directional Gal1/10 promotor, allowing the simultaneous expression of both subunits from one plasmid.

### Recombinant Synthesis of PreColD and PreColP

Recombinant synthesis of preColD and preColP, two major protein components of Mussel Byssus is an example of the present invention. The c-DNA of PreColP and PreColD in E. coli cloning vectors has been obtained from Prof. Waite (UCSB, USA). The cDNA is amplified by PCR and cloned into different yeast expression vectors. The vectors differ in copy number per cell, as well as in the choice of the activator (either the constitutive promotor GPD or the inducible promotor GAL4). Also the original signal sequence will be replaced by the signal sequence of the yeast α-MF for maximum localization efficiency.

### Detection of ColP and ColD During Recombinant Synthesis

The test for the efficient recombinant synthesis of mussel collagen requires availability of polyclonal antibodies against mussel collagen. Preliminary tests with polyclonal antibodies against human collagen type I-III showed a very weak cross-reactivity against chemically denatured collagen from mussel byssus. This cross-reactivity is not sufficient to detect the levels of collagen present during the recombinant synthesis. Hence antibodies need to be raised against mussel collagen. In order to be able to raise antibodies, purified native mussel collagen is required. Byssus will be extracted from fresh mussels and purified using several chromatographic methods (reverse phase chromatography among others).

The purified protein samples, which contain both preColD and preColP, are used to immunize rabbits and generate antibodies.

### Biophysical Studies on Recombinant Collagen

Various physical methods can be used to characterize the individual proteins preColP/preColD and to evaluate the efficiency of fiber formation on self-assembly. These methods include far- and near-UV circular dichroism (CD), static and dynamic light scattering, fourier transformed infrared spectroscopy (FTIR), electron microscopy (EM), atomic force microscopy (AFM) and field flow fractionation (FFF).

### Characterization of Individual PreColP and PreColD

CD and FTIR will be used to determine the secondary and tertiary structures of preColP and preColD. Their chemical and thermal stability will also be tested under various conditions. Data on the shape of the proteins involved in the collagen formation are provided by light scattering, by AFM and TEM.

### Evaluation of Rate and Efficiency of Fiber Formation

Secondary and tertiary structure of mussel byssus collagen are analyzed by CD and FTIR. AFM and EM will provide information on the quaternary structure and morphology of the assembled aggregates and fibers. FFF, a one-phase matrix-free chromatography, would be used to evaluate the different kinds of species formed during assembly of the collagen. Since FFF is a matrix-free chromatography technique, it can separate different dissolved macromolecules, especially fibers, which can not be separated by other classical chromatographic techniques.

Kinetics of the assembly process will also be investigated with CD and FTIR, which can be performed as a function of time by monitoring changes in the secondary and tertiary structure during the fiber formation. Further, static light scattering, dynamic light scattering and time-lapse AFM allow to monitor protein assembly in real-time.

Fluorescent dyes can also be used to investigate the structural changes associated with protein assembly. The fluorescent properties of some dyes, such as the N-benzyl derivatives of 3-chloro-6-methoxy-9 aminoacridine and amino naphthalene sulfonic acids, change with the polarity of the protein environment. Therefore, labeling of collagen with these dyes are used to study its assembly process.

### Study of the Role of Metals in the Assembly and Cross-Linking of Collagens

### Role of GGH in DOPA and Tyrosine Cross-Links

The amino acid sequence GGH has been observed at the carboxylterminus of both preColP and preColD. The tripeptide NH2-Gly-Gly-His-COOH(GGH) mediates cross-linking of associated proteins in solution in the presence of nickel acetate [Ni(OAc)2] and oxidant magnesium monoperoxyphthalate (MMPP) [18, 19]. Further, the peptide provides a favorable coordination environment for the nickel center, and a putative Ni(III) intermediate is thought to abstract an electron from the aromatic ring of an accessible tyrosine, leading to a tyrosyl radical after the loss of a proton (see FIG. 5). The highly activated radical intermediate couples to a nearby tyrosine leading to a cross-linked adduct.

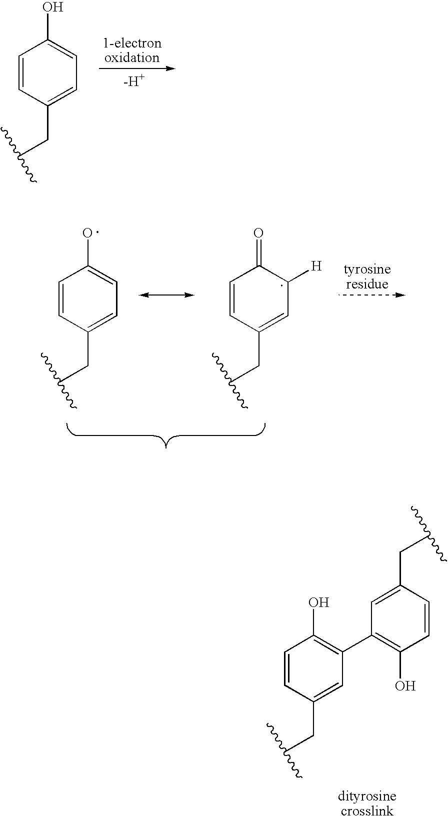

### Mechanism of Tyrosine Cross-Linking

A possible role for the GGH in the carboxylterminus of mussel collagens could be to bind Ni(II) in order to form the active catalyst Ni-GGH. This complex can slowly catalyze aerial oxidation of tyrosine and DOPA to form cross-links. The proximity of the catalyst to tyrosine and/or DOPA would significantly increase the oxidation rates. To test this hypothesis, the GGH sequence could be genetically deleted or modified so that it would not bind nickel. The rate of cross-linking and assembly would be monitored by methods described above.

### Chemical Oxidation of Tyrosines to Form Cross-Links in Collagen

Visible-light irradiation in the presence of ruthenium(II) tris(bipyridyl) dication [Ru(bpy)₃²⁺] and an electron acceptor such as ammonium persulfate (APS) [18, 20, 21] induces very efficient cross-linking between contacting proteins. This process is highly efficient and the mechanism has been assumed to be similar to that of Ni/GGH/MMPP. The fiber formed from self-assembly of preColP and/or preColD will be subject to irradiation with [Ru(bpy)₃²⁺] in the presence of APS. This should lead to increased cross-linking of tyrosine/DOPA in byssal collagen and lead to fibers with altered mechanical properties, which will be assessed upon physico-chemical characterization as described above.

### Further Examples and Sequences:

DNA sequences of mussel collagen preColP and proColD are provided in the following. The cDNA of both preCol proteins (P and D) were integrated in the pGEM-T cloning vectors. In order to verify the starting material, both cDNAs were completely sequenced and as standard primers T7 and SP6 were used and as internal primers preCol (P or D)-T7/1 and SP6/1, respectively, were used. The obtained DNA sequences showed differences as regards the published versions of both preCols and were compared accordingly.

preColP
(SEQ ID NO:1)
atggttcg gttctcccta gcatcggtac tattactggc agtcaccagc acagctttcg
ctggaccagt tagtgattat ggtggtggtg gaatcaaagt agtaccctac cacggaggcg
gaggtggaag cggcggcggt ggcggtggag gccatggcgg aagcggtatt ggtggtatcg
gaggaggatc atcacatgca catgcccact cttcagcatc tgcccatgtg caccattttg
gaccaggtgg atcttcacac gcatcagctg gttcatcatc ccatgcatcc gcatcccata
acggtttagg aggtggcagt gctcatgcac atagcagttc cagcgccaac gctcattccg
gtggattcgg tggattcggc ggtattggtg gtattggcgg tattggccca ggaggaagtg
tcggaggcgg tattggccca ggaggaagtg tcggaggcgg cattggcggt attggcggta
ttggcggcgg tggtggacca ggcggtaatg gcggtatcgg attcggacca ggattcggag
gaggattcgg accaggttca tctgctagtg gatccggaag tggcagcgca ttcggtggtc
caggaggttc aagcgcaagc gcaaacgcag ctgcacgtgc aaatgcaaat ggtggtggag
gattcggtgg accaggtacc ccaggaaact caggaccacc aggccaaccc ggactaccag
gagcaccagg ccaaccagga cgtccaggaa gtaccccacc aggtcgacca ggaaaccccg
gaccaccagg tcaaccaggt aacccaggac gtccaggctc ttcaggaaga ccaggaggat
ccggccaacc aggaggtcca ggacgtccag gaacccccgg caaaccagga aaccgaggac
aaccaggaca gccaggcggc ccaggacaac caggtcaccc aggagcagga ggacaaccag
gacgaaacgg aaatccagga aaccccggta aaccaggaac accaggtcac ccaggaacag
caggatcacg aggaatgcca ggaaccccag gaaccccagg acaaccagga attccaggca
ccgtcggagg acgaggacca agaggaccag ctggaatcat cggattaatt ggaccaaaag
gaaatccagg agagccagga aatccaggtg caccaggagg cccaggatct acaggaccac
aaggaccaca aggaccagcc ggaggaccag gagcatcagg cggaccagga gacaaaggcg
caccaggtac accaggagga actggaccaa gaggaccaat cggaccatca ggaccatcag
gagcaccagg ggaccaagga ccacaaggag gtagaggaac accaggactc gcaggcaaac
caggacctaa aggactacaa ggatcaaatg gagaagttgg accccaagga ccatctggac
ccgcaggacc acaaggccca caaggaaaga acggtgtcaa aggagcagca ggagatcaag
gagctagggg accagaagga aaagccggac cagctggacc acaaggagaa acaggaccaa
aaggaccaac aggagcacaa ggaccagccg gtccagccgg accatcagga gaacaaggac
caggagggga aagaggaggc cagggaccac aaggagctga aggaccaagt ggaccagcag
gaccaagagg accagcagga tcacaaggac caagtggtga acgcggagaa ccaggagcac
caggtaaaaa aggaccaaat ggagaccgag gaaaccaagg atcaccagga gcaccaggca
aaaacggagc acgaggaaat agaggatcaa gaggaagcaa cggatcaccc ggcagatcag
gatcaccagg aagccgagga aaaccaggac cacaaggacc acatggacca agaggagcaa
gaggatcacc aggacaaaaa ggaccacgtg gagaccaagg agcaccaggt gttattcgta
ttgttatcga tgaccagaga acaggaccag aagttgcaga attcccagga tttggtggat
tcggaggagc ttcagctaac gcagcaagtt cagcaaatgc atttgctggt ggacccggtg
gttccgctgg agcaggttca tcatcaggag ctaacgcaaa cgcaggtgga ttcccattcg
gaggaggacc attcggagga gcaggaggtg gtcccggagc agcaggaggc ccaggaggag
caggaggccc aggaggagta ggaggaggag ttggaggtgg accaggagga gtaggaggtg
gagtaggagg tggaccagga ggagtaggag gtggaccagg aggagcagga ccaggaggag
caggaggatt tggaccagga ggagcaggag gatttggtgg atttggagga ggatctagcg
ctggagcatc atcatcagga tcagcatctg catctaacgg tggaccattc ggagtactca
atgtaggacc cggaggtaga atcggtggtg gaagcgcatc agcatctgca gcatctagag
cacatgcaca cgcttttggt ggtctcggag ggggaagtgc ctcagctggt agtcattcct
catctagctc acactcattt ggcggacacg tattccacag tgtgacccat catggaggtc
catcacatgt ttcaagcgga ggtcacggag gtcatggagg aggtccatac aaacctggat attaa

Due to the degeneration of the genetic code, not every base exchange is leading to an amino acid exchange. Therefore, the DNA sequences were translated into the amino acid sequences and were compared. Here, the alignment of the published sequence (COYNE et al., 1997) (variant P38, (COYNE & WAITE, 2000)) with the sequence of preColP obtained by sequencing is shown. The database sequence corresponds to SEQ ID NO: 9, the sequenced preColP sequence is SEQ ID NO:3.

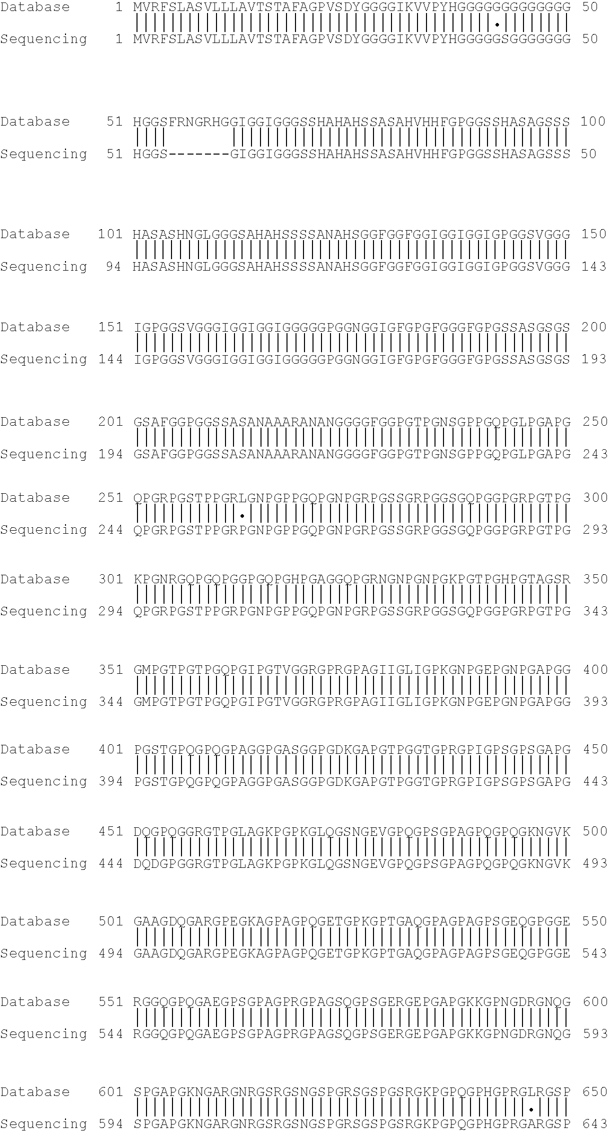

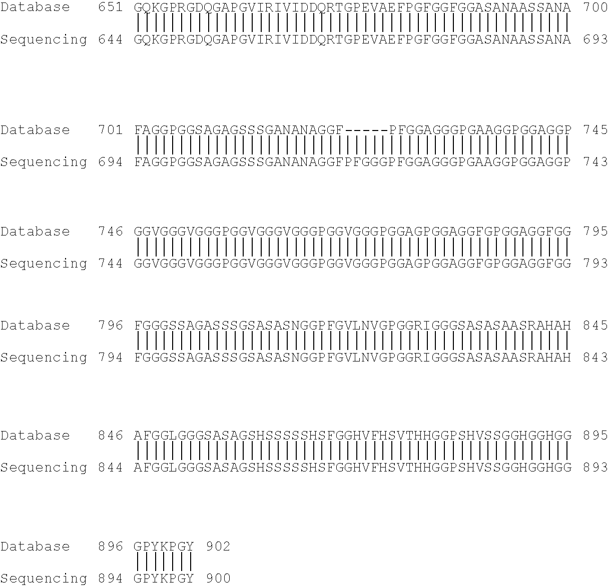

COYNE & WAITE already showed the existence of different preColP variants (P22, P33 and P38) in certain partial regions of their cDNA sequence (COYNE & WAITE, 2000). If these short, known sequence regions of variant P22 are compared with the present DNA sequence of preColP, a matching of 100% is achieved.

preColD
(SEQ ID NO:2)
atggtcta caaactcctg accgtgtgtc ttgtagcatc tcttctagag atttgcttag
ctgactataa cggcaacaaa cagtatggcg gcagatacgg caacagatac ggaaacggtt
taggaggcgg taatggtggt gcaggagccg tagcccatgc ccatgcccat gcccatgcca
gtgccggagc aaacggaaga gcaagagcac atgcacgagc cttggcccat gcacatgccg
gtggtggcgc tgcacatgga cacccaggat tcccagttgg tggtagcgca agcgcagccg
cacgagcagc agcacgagca tcagcaggag gattaggtgg attcggatca gcagcagcca
atgcagcagc agcagcaaga gcaggagcag gatttggtgg attcggtgga ttaggaggat
tcggaggact cggaggagtt ggcggtccag gtcaaccagg acatgccggt aaacacggaa
ccgcaggagc agcaggcaaa gcaggacgtc caggaccatg tggagataga ggggcaccag
gagtaccagg caaacaagga ccagtaggag gacaaggacc agcaggacca cgaggaccac
gaggagatga aggaccagtt ggaccaaagg gcgaaccagg agcaagagga gctgatggta
aaccaggaga caaaggacct gatggagaaa ccggaccaca aggaccagct ggaccaaagg
gacaagtagg agaccaaggc aaaccaggag caaagggaga aaccggagat caaggagcac
gaggtgaagc aggaaaggcc ggcgaacaag gaccaggagg catccaagga ccaaagggac
cagtaggagg acaaggacca gcaggaccag ccggaccact cggaccacaa ggaccaatgg
gtgaacgagg accacaagga ccaacaggat cagaaggacc agttggagca ccaggaccaa
agggatcagt cggagaccaa ggagcacaag gagaccaagg agcaactggc gctgatggca
aaaagggaga accaggagag agaggacaac aaggagcagc aggaccagtc ggccgaccag
gaccaagagg agatagagga gcaaagggaa ttcaaggaag ccgaggacga ccaggtggta
tgggtagacg aggaaaccgt ggatcccaag gagcagtagg accacgagga gaaactggcc
cagacggtaa ccaaggacaa cgtggagaac aaggagcacc aggagttatc acccttgtca
ttgaagacct cagaacagcc ggagtagaaa gccccgtaga aacctttgac gcaggagcag
gaaccggtgg accagcacca ggagtaggag cagcagcaac agcaggagca tttgcaggag
caggaccagg aggagctaat gcaggaggaa acgcagccgc aggagcagga ccaggagtag
gaccaggagg actcggagga ctaggaggac ttggtgcagg tggactcgga ggtggactcg
gcggtggact cggaggatta ggaggagcag gaggtttagg tggtggactc ggaggattag
gaggaggttt aggtggtgga ctcggaggtt taggaggtgg agcaggagga gcaggaggcg
caggagcagg aggaaacggt ggagcaggag caggaggagc aggaggaaac ggtggaggat
cagccgcagc acgagcagca gcacaagcag cagcagcagc aggaggaaac ggtggagcag
cacaagcagc agcacaagca gcagcatcag cagcagcaaa ttcaggactt ggagcaggag
cagcaagagc agcagcatca gcagccgcta gagcaaccgt agcaggacat ggaagtggaa
ccgccgcagc agcagccaac gcagccgcac aagcacatgc agcaacacga ggacaaggag
gatcacacgc acacgctgcc gccgcagctc acgcagccgc aagtagcgta atccatggtg
gtgactatca cggaaacgat gccggctatc acaaaccagg atattaa

In the following, the alignment of the published sequence (QIN et al., 1997) [gi:2772914] with the overall sequence obtained by sequencing of preColD is shown. The DNA sequences were translated into the protein sequence. It is noted that the database sequence is SEQ ID NO: 8 and the sequence obtained by sequencing is SEQ ID NO: 4.

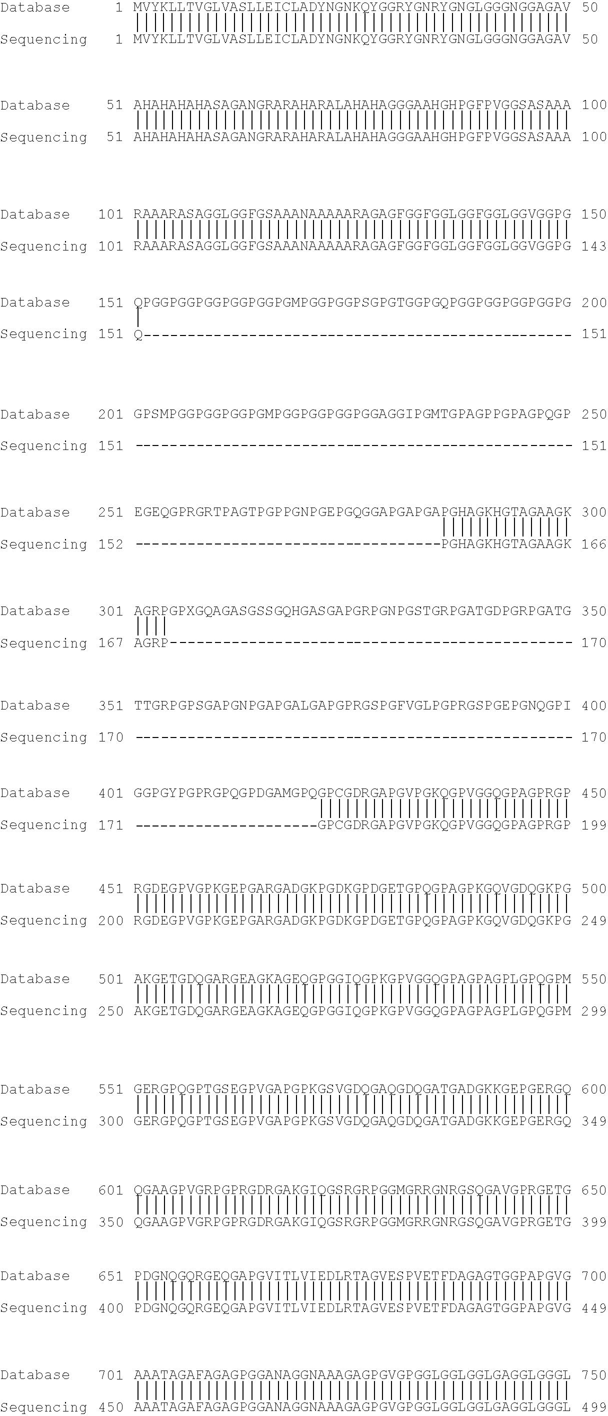

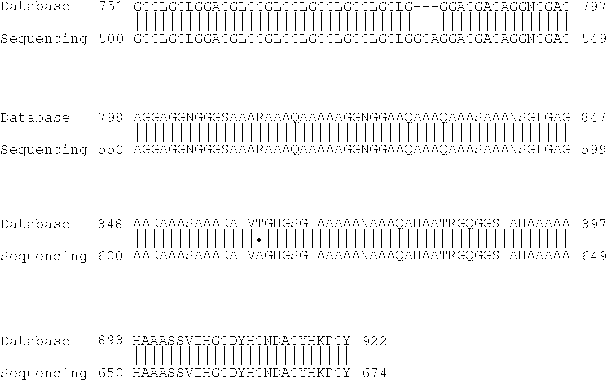

There are significant differences in both sequences: The preColD sequence used is by 250 amino acids shorter than the published sequence. The major part of the amino acids in the collagen domain is missing. Therefore, the presently disclosed preColD gene is an up to now unpublished and unknown version of the preColD gene. It is noted that the truncation of the collagen domain increases the amount of silk fibroin domains in the whole protein and therefore, the behavior of the overall protein will be different.

### Expression Construct of P4H

In the following, the DNA sequence after expression plasmid for P4H in the region of SacII to ApaI is shown as double strand. The beginning and the end of MFa/P4H fusion constructs are both printed. The used restriction sites are underligned.

```
SEQ ID NO: 7
      1 ccgcggtcat tacagttcat ctttcacagc tttctgatca tcgtcttcct ccatgtctgg
      1 ggcgccagta atgtcaagta gaaagtgtcg aaagactagt agcagaagga ggtacagacc
        SacII 2 × Stop
    61 ctcctctgct tcttccaggt cctcgagatc gtcatcatcc cctgccccat cctggccacc
    61 gaggagacga agaaggtcca ggagctctag cagtagtagg ggacggggta ggaccggtgg
  121 cgagaggtcc ttaaagaatt ttggtaqgtc gcacgcaagg ggcaacatta gttactggca
  121 gctctccagg aatttcttaa aaccatccag cgtgcgttcc ccgttgtaat caatgaccgt
  181 cctgtcggca ctggcaggaa agaacttgag tgtggggaag ctgtgcactt tgacggcctc
  181 ggacagccgt gaccgtcctt tcttgaactc acaccccttc gacacgtgaa actgccggag
  241 cacctcgttg gcagtcgagt ccatcttggc gatgacgatg ttctcatggt ccttgtacgt
  241 gtggagcaac cgtcagctca ggtagaaccg ctactgctac aagagtacca ggaacatgca
  301 ctctcccagt ttatcccaaa tgggagccaa ctgtttgcag tgaccacacc atggggcata
  301 gagagggtca aatagggttt accctcggtt gacaaacgtc actggtgtgg taccccgtat
  361 gaactccaca aagacgtttt ttttctcatc aaaagccacg tcttcaaagt tcttcccaac
  361 cttgaggtgt ttctgcaaaa aaaagagtag ttttcggtgc agaagtttca agaagggttg
  421 aagcaccttg acaggctgct tgtcccagtc ctccggcagc tcctggctca tcaggtgggg
  421 ttcgtggaac tgtccgacga acagggtcag gaggccgtcg aggaccgagt agtccacccc
  481 cttgattttg ccctccagga agcggtggca gaactctgtg atcctctctg ccgtcagctc
  481 gaactaaaac gggaggtcct tcgccaccgt cctgagacac taggagagac ggcagtcgag
  541 ctccgattcg ggcttgtact tggtcatctc ctcctccagg gtgatgaggc gcacggccgg
  541 gaggctaagc ccgaacatga accagtagag gaggaggtcc cactactccg cgtgccggcc
  601 gcactcttcc ttcttcaggc caaagaactc gaggatgcgc tggttgtcgg tgtggtcgct
  601 cgtgagaagg aagaagtccg gtttcttgag ctcctacgcg accaacagcc acaccagcga
  661 gtcgatgaag atgaacagga tcttgccctt gaagctctcg gctgctgttt tgaagttgct
  661 cagctacttc tacttgtcct agaacgggaa cttcgagagc cgacgacaaa acttcaacga
  721 cagtttgccg tcatagtcag acacactctt gggcaagaac agcaggatgt gagtcttgat
  721 gtcaaacggc agtatcagtc tgtgtgagaa cccgttcttg tcgtcctaca ctcagaacta
  781 ttcacctcca aaaatcttcg gggctgtctg ctcggtgaac tcgatgacaa ggggcagctg
  781 aagtggaggt ttttagaagc cccgacagac gagccacttg agctactgtt ccccgtcgac
  841 gttgtgtttg ataaagtcca gcaggttctc cttggtgacc tccccttcaa agttgttccg
  841 caacacaaac tatttcaggt cgtccaagag gaaccactgg aggggaagtt tcaacaaggc
  901 gccttcatca aacttcttaa agaggacaac cccatctttg tcgagctggt atttggagaa
  901 cggaagtagt ttgaagaatt tctcctgttg gggtagaaac agctcgacca taaacctctt
  961 cacgtcactg ttggaagtga tcccaaatgg tatgtcatcg atggcctctg ctgcctgcaa
  961 gtgcagtgac aaccttcact agggtttacc atacagtagc taccggagac gacggacgtt
1021 aaactgcttg gcagagtccg actccacgtc cttgaagaag ccgatgacag ccacctcgct
1021 tttgacgaac cgtctcaggc tgaggtgcag gaacttcttc ggctactgtc ggtggagcga
1081 ggactccacc aaggactctg cagctgcgcc gtcaggcagg gtggtggcag ccgggcccgt
1081 cctgaggtgg ttcctgagac gtcgacgcgg cagtccgtcc caccaccgtc ggcccgggca
                                                                ApaI
1141 gcgcttcttc agccagttca cgatgtcatc agcctctctg ccagctgtat attccttggg
1141 cgcgaagaag tcggtcaagt gctacagtag tcggagagac ggtcgacata taaggaaccc
1201 ggaagccgtg tctccattcc tgaagaactt gatggtggga tagccgcgca cgccgtactg
1201 ccttcggcac agaggtaagg acttcttgaa ctaccaccct atcggcgcgt gcggcatgac
1261 ctgggccagg tcagactcct ccgtggcgtc caccttggcc aacctgatct cggaaccttc
1261 gacccggtcc agtctgagga ggcaccgcag gtggaaccgg ttggactaga gccttggaag
1321 tgccttcagc ttcccagcgg ctttggcata ctcaggggcc agagccttgc agtggccaca
1321 acggaagtcg aagggtcgcc gaaaccgtat gagtccccgg tctcggaacg tcaccggtgt
1381 ggttccccgt atcttgaggt ggtcgtccat gaacacccgg cggtcgcgga ggcgcttcaa
1381 ccaaggggca tagaactcca ccagcaggta cttgtgggcc gccagcgcct ccgcgaagtt
1441 gcttttccgc agcaccagga cgtggtcctc ctcctccgga gcgtcagcta atgcggagga
1441 cgaaaaggcg tcgtggtcct gcaccaggag gaggaggcct cgcagtcgat tacgcctcct
                                            BspEI
1501 tgctgcgaat aaaactgcag taaaaattga aggaaatctc atggatccgg ggttttttct
1501 acgacgctta ttttgacgtc atttttaact tcctttagag tacctaggcc ccaaaaaaga
                                                  Start BamHI
1561 ccttgacgtt aaagtataga ggtatattaa caattttttg ttgatacttt tattacattt
1561 ggaactgcaa tttcatatct ccatataatt gttaaaaaac aactatgaaa ataatgtaaa
1621 gaataagaag taatacaaac cgaaaatgtt gaaagtatta gttaaagtgg ttatgcagtt
1621 cttattcttc attatgtttg gcttttacaa ctttcataat caatttcacc aatacgtcaa
1681 tttgcattta tatatctgtt aatagatcaa aaatcatcgc ttcgctgatt aattacccca
1681 aaacgtaaat atatagacaa ttatctagtt tttagtagcg aagcgactaa ttaatggggt
1741 gaaataaggc taaaaaacta atcgcattat catcctatgg ttgttaattt gattcgttca
1741 ctttattccg attttttgat tagcgtaata gtaggatacc aacaattaaa ctaagcaagt
1801 tttgaaggtt tgtggggcca ggttactgcc aatttttcct cttcataacc ataaaagcta
1801 aaacttccaa acaccccggt ccaatgacgg ttaaaaagga gaagtattgg tattttcgat
1861 gtattgtaga atctttattg ttcggagcag tgcggcgcga ggcacatctg cgtttcagga
1861 cataacatct tagaaataac aagcctcgtc acgccgcgct ccgtgtagac gcaaagtcct
1921 acgcgaccgg tgaagacgag gacgcacgga ggagagtctt ccttcggagg gctgtcaccc
1921 tgcgctggcc acttctgctc ctgcgtgcct cctctcagaa ggaagcctcc cgacagtggg
1981 gctcggcggc ttctaatccg tacttcaata tagcaatgag cagttaagcg tattactgaa
1981 cgagccgccg aagattaggc atgaagttat atcgttactc gtcaattcgc ataatgactt
2041 agttccaaag agaaggtttt tttaggctaa gataatgggg ctctttacat ttccacaaca
2041 tcaaggtttc tcttccaaaa aaatccgatt ctattacccc gagaaatgta aaggtgttgt
2101 tataagtaag attagatatg gatatgtata tggatatgta tatggtggta atgccatgta
2101 atattcattc taatctatac ctatacatat acctatacat ataccaccat tacggtacat
2161 atatgattat taaacttctt tgcgtccatc caaaaaaaaa gtaagaattt ttgaaaattc
2161 tatactaata atttgaagaa acgcaggtag gttttttttt cattcttaaa aacttttaag
2221 aaggaattcg atatcaagct tatcgatacc gtcgacatga gatttccttc aatttttact
2221 ttccttaagc tatagttcga atagctatgg cagctgtact ctaaaggaag ttaaaaatga
          EcoRI SalI Start
2281 gcagttttat tcgcagcatc ctccgcgcta gctcatccag gcttttttac ttcaattggt
2281 cgtcaaaata agcgtcgtag gaggcgcgat cgagtaggtc cgaaaaaatg aagttaacca
                                  NheI
2341 cagatgactg atttgatcca tactgagaaa gatctggtga cttctctgaa agattatatt
2341 gtctactgac taaactaggt atgactcttt ctagaccact gaagagactt tctaatataa
2401 aaggcagaag aggacaagtt agaacaaata aaaaaatggg cagagaagtt agatcggcta
2401 ttccgtcttc tcctgttcaa tcttgtttat ttttttaccc gtctcttcaa tctagccgat
2461 actagtacag cgacaaaaga tccagaagga tttgttgggc atccagtaaa tgcattcaaa
2461 tgatcatgtc gctgttttct aggtcttcct aaacaacccg taggtcattt acgtaagttt
2521 ttaatgaaac gtctgaatac tgagtggagt gagttggaga atctggtcct taaggatatg
2521 aattactttg cagacttatg actcacctca ctcaacctct tagaccagga attcctatac
2581 tcagatggct ttatctctaa cctaaccatt cagagaccag tactttctaa tgatgaagat
2581 agtctaccga aatagagatt ggattggtaa gtctctggtc atgaaagatt actacttcta
2641 caggttgggg cagccaaagc tctgttacgt ctccaggata cctacaattt ggatacagat
2641 gtccaacccc gtcggtttcg agacaatgca gaggtcctat ggatgttaaa cctatgtcta
2701 accatctcaa agggtaatct tccaggagtg aaacacaaat cttttctaac ggctgaggac
2701 tggtagagtt tcccattaga aggtcctcac tttgtgttta gaaaagattg ccgactcctg
2761 tgctttgagt tgggcaaagt ggcctataca gaagcagatt attaccatac ggaactgtgg
2761 acgaaactca acccgtttca ccggatatgt cttcgtctaa taatggtatg ccttgacacc
2821 atggaacaag ccctaaggca actggatgaa ggcgagattt ctaccataga taaagtctct
2821 taccttgttc gggattccgt tgacctactt ccgctctaaa gatggtatct atttcagaga
2881 gttctagatt atttgagcta tgcggtatat cagcagggag acctggataa ggcacttttg
2881 caagatctaa taaactcgat acgccatata gtcgtccctc tggacctatt ccgtgaaaac
2941 ctcacaaaga agcttcttga actagatcct gaacatcaga gagctaatgg taacttaaaa
2941 gagtgtttct tcgaagaact tgatctagga cttgtagtct ctcgattacc attgaatttt
3001 tattttgagt atataatggc taaagaaaaa gatgtcaata agtctgcttc agatgaccaa
3001 ataaaactca tatattaccg atttcttttt ctacagttat tcagacgaag tctactggtt
3061 tctgatcaga aaactacacc aaagaaaaaa ggggttgctg tggattacct gccagagaga
3061 agactagtct tttgatgtgg tttctttttt ccccaacgac acctaatgga cggtctctct
3121 cagaagtacg aaatgctgtg ccgtggggag ggtatcaaaa tgacccctcg gagacagaaa
3121 gtcttcatgc tttacgacac ggcacccccc ccatagtttt actggggagc ctctgtcttt
3181 aaactctttt gccgctacca tgatggaaac cgtaatccta aatttattct ggctccagct
3181 tttgagaaaa cggcgatggt actacctttg gcattaggat ttaaataaga ccgaggtcga
3241 aaacaggagg atgaatggga caagcctcgt attattcgct tccatgatat tatttctgat
3241 tttgtcctcc tacttaccct gttcggagca taataagcga aggtactata ataaagacta
3301 gcagaaattg aaatcgtcaa agacctagca aaaccaaggc tgagccgagc tacagtacat
3301 cgtctttaac tttagcagtt tctggatcgt tttggttccg actcggctcg atgtcatgta
3361 gaccctgaga ctggaaaatt gaccacagca cagtacagag tatctaagag tgcctggctc
3361 ctgggactct gaccttttaa ctggtgtcgt gtcatgtctc atagattctc acggaccgag
3421 tctggctatg aaaatcctgt ggtgtctcga attaatatga gaatacaaga tctaacagga
3421 agaccgatac ttttaggaca ccacagagct taattatact cttatgttct agattgtcct
3481 ctagatgttt ccacagcaga ggaattacag gtagcaaatt atggagttgg aggacagtat
3481 gatctacaaa ggtgtcgtct ccttaatgtc catcgtttaa tacctcaacc tcctgtcata
3541 gaaccccatt ttgactttgc acggaaagat gagccagatg ctttcaaaga gctggggaca
3541 cttggggtaa aactgaaacg tgcctttcta ctcggtctac gaaagtttct cgacccctgt
3601 ggaaatagaa ttgctacatg gctgttttat atgagtgatg tgtctgcagg aggagccact
3601 cctttatctt aacgatgtac cgacaaaata tactcactac acagacgtcc tcctcggtga
3661 gtttttcctg aagttggagc tagtgtttgg cccaaaaaag gaactgctgt tttctggtat
3661 caaaaaggac ttcaacctcg atcacaaacc gggttttttc cttgacgaca aaagaccata
3721 aatctgtttg ccagtggaga aggagattat agtacacggc atgcagcctg tccagtgcta
3721 ttagacaaac ggtcacctct tcctctaata tcatgtgccg tacgtcggac aggtcacgat
3781 gttggcaaca aatgggtatc caataaatgg ctccatgaac gtggacaaga atttcgaaga
3781 caaccgttgt ttacccatag gttatttacc gaggtacttg cacctgttct taaagcttct
3841 ccttgtacgt tgtcagaatt ggaatgatga gggccc
3841 ggaacatgca acagtcttaa ccttactact cccggg
                                  2 × Stop ApaI
```

### Protein Sequences

MFa-P4HA

(SEQ ID NO:5)

MRFPSIFTAV LFAASSALAH PGFFTSIGQM TDLIHTEKDL
VTSLKDYIKA EEDKLEQIKK WAEKLDRLTS TATKDPEGFV
GHPVNAFKLM KRLNTEWSEL ENLVLKDMSD GFISNLTIQR
PVLSNDEDQV GAAKALLRLQ DTYNLDTDTI SKGNLPGVKH
KSFLTAEDCF ELGKVAYTEA DYYHTELWME QALRQLDEGE
ISTIDKVSVL DYLSYAVYQQ GDLDKALLLT KKLLELDPEH
QRANGNLKYF EYIMAKEKDV NKSASDDQSD QKTTPKKKGV
AVDYLPERQK YEMLCRGEGI KMTPRRQKKL FCRYHDGNRN
PKFILAPAKQ EDEWDKPRII RFHDIISDAE IEIVKDLAKP
RLSRATVHDP ETGKLTTAQY RVSKSAWLSG YENPVVSRIN
MRIQDLTGLD VSTAEELQVA NYGVGGQYEP HFDFARKDEP
DAFKELGTGN RIATWLFYMS DVSAGGATVF PEVGASVWPK
KGTAVFWYNL FASGEGDYST RHAACPVLVG NKWVSNKWLH
ERGQEFRRPC TLSELE
MFa-P4HB

(SEQ ID NO:6)

MRFPSIFTAV LFAASSALAD APEEEDHVLV LRKSNFAEAL
AAHKYLLVEF YAPWCGHCKA LAPEYAKAAG KLKAEGSEIR
LAKVDATEES DLAQQYGVRG YPTIKFFRNG DTASPKEYTA
GREADDIVNW LKKRTGPAAT TLPDGAAAES LVESSEVAVI
GFFKDVESDS AKQFLQAAEA IDDIPFGITS NSDVFSKYQL
DKDGVVLFKK FDEGRNNFEG EVTKENLLDF IKHNQLPLVI
EFTEQTAPKI FGGEIKTHIL LFLPKSVSDY DGKLSNFKTA
AESFKGKILF IFIDSDHTDN QRILEFFGLK KEECPAVRLI
TLEEEMTKYK PESEELTAER ITEFCHRFLE GKIKPHLMSQ
ELPEDWDKQP VKVLVGKNFE DVAFDEKKNV FVEFYAPWCG
HCKQLAPIWD KLGETYKDHE NIVIAKMDST ANEVEAVKVH
SFPTLKFFPA SADRTVIDYN GERTLDGFKK FLESGGQDGA
GDDDDLEDLE EAEEPDMEED DDQKAVKDEL
Sequence of MFa

(SEQ ID NO:10)

MRFPSIFTAV LFAASSALA

### REFERENCES

1. Yonge, M. (1962). On the significance of the byssus in the bivalvia and its effects in evolution. J. Mar. Biol. Ass. U. K. 42, 113-125.
2. Qin, X.-X., Coyne, K. J., and Waite, J. H. (1997). Tough tendons. Mussel byssus has collagen with silk-like domains. Journal of Biological Chemistry 272, 32623-32627.
3. Waite, J. H. (1992). Results Probl. Cell Differ. 19, 27.
4. Qin, X.-X., and Waite, J. H. (1995). Exotic Collagen Gradients in the Byssus of ht eMussel Mytilus Edulia. The Journal of Experimental Biology 198, 633-644.
5. Vaccaro, E., and Waite, J. H. (2001). Yield and Post-Yield Behavior of Mussel Byssal Thread: A Self-Healing Biomolecular Material. Biomacromolecules 2, 906-911.
6. Coyne, K. J., Qin, X.-X., and Waite, J. H. (1997). Extensible collagen in mussel byssus: a natural block copolymer. Science (Washington, D.C.) 277, 1830-1832.
7. Waite, J. H., Qin, X.-X., and Coyne, K. J. (1998). The peculiar collagens of mussel byssus. Matrix Biology 17, 93-106.
8. Coombs, T. L., and Keller, P. J. (1981). Mytilus byssal threads as an environmental marker for metal ions. Aquat. Toxicol. 1981, 291-300.
9. Swann, C. P., Adewole, T., and Waite, J. H. (1998). Preferential manganese accumulation in dreissenid byssal threads. Comparative Biochemistry and Physiology, Part B: Biochemistry & Molecular Biology 119B, 755-759.
10. Taylor, S. W., Chase, D. B., Emptage, M. H., Nelson, M. J., and Waite, J. H. (1996). Ferric Ion Complexes of a DOPA-Containing Adhesive Protein from Mytilus edulis. Inorganic Chemistry 35, 7572-7577.
11. Sun, C., Vaccaro, E., and Waite, J. H. (2001). Oxidative stress and the mechanical properties of naturally occurring chimeric collagen-containing fibers. Biophysical Journal 81, 3590-3595.
12. Myllyharju, J., Nokelainen, M., Vuorela, A., and Kivirikko, K. I. (2000). Expression of recombinant human I-III collagens in the yeast Pichia pastoris. Biochem. Soc. Trans. 28, 353-357.
13. Prockop, D. J., and Kivirikko, K. I. (1995). Annu. Rev. Biochem. 64, 403-434.
14. Olsen, D. R., Leigh, S. D., Chang, R., McMullin, H., Ong, W., Ernest, T., Chisholm, G., Birk, D. E., Berg, R. A., Hitzeman, R. A., and Toman, P. D. (2001). Production of Human Type 1 Collagen in Yeast Reveals Unexpected New Insights into Molecular assembly of Collagen Trimers. J. Biol. Chem. 276, 24038-24043.
15. Scheibel, T. (2004). Spider silks: recombinant synthesis, assembly, spinning, and engineering of synthetic proteins. Microbial Cell Factories 3, No pp. given.
16. Huemmerich, D., Scheibel, T., Vollrath, F., Cohen, S., Gat, U., and Ittah, S. (2004). Novel Assembly Properties of Recombinant Spider Dragline Silk Proteins. Current Biology 14, 2070-2074.
17. Huemmerich, D., Helsen, C. W., Quedzuweit, S., Oschmann, J., Rudolph, R., and Scheibel, T. (2004). Primary Structure Elements of Spider Dragline Silks and Their Contribution to Protein Solubility. Biochemistry 43, 13604-13612.
18. Brown, K. C., and Kodadek, T. (2001). Protein cross-linking mediated by metal ion complexes. Metal Ions in Biological Systems 38, 351-384.
19. Fancy, D. A., Denison, C., Kim, K., Xie, Y., Holdeman, T., Amini, F., and Kodadek, T. (2000). Scope, limitations and mechanistic aspects of the photo-induced cross-linking of proteins by water-soluble metal complexes. Chemistry & Biology 7, 697-708.
20. Burdine, L., Gillette, T. G., Lin, H.-J., and Kodadek, T. (2004). Periodate-Triggered Cross-Linking of DOPA-Containing Peptide-Protein Complexes. Journal of the American Chemical Society 126, 11442-11443.
21. Kim, K., Fancy, D. A., Carney, D., and Kodadek, T. (1999). Photoinduced Protein Cross-Linking Mediated by Palladium Porphyrins. Journal of the American Chemical Society 121, 11896-11897.

### Further References

Brake, A. J. (1990) Alpha-factor leader-directed secretion of heterologous proteins from yeast. Methods Enzymol. 185: 408-21
Bulleid, N. J., John, D. C. & Kadler, K. E. (2000) Recombinant expression systems for the production of collagen. Biochem. Soc. Trans. 28: 350-3
Coyne, K. J. & Waite, J. H. (2000) In search of molecular dovetails in mussel byssus: from the threads to the stem. J. Exp. Biol. 203: 1425-31
Keizer-Gunnink, I., Vuorela, A., Myllyharju, J., Pihlajaniemi, T., Kivirikko, K. I. & Veenhuis, M. (2000) Accumulation of properly folded human type III procollagen molecules in specific intracellular membranous compartments in the yeast Pichia pastoris. Matrix Biol. 19: 29-36
Lucas, J. M., Vaccaro, E. & Waite, J. H. (2002) A molecular, morphometric and mechanical comparison of the structural elements of byssus from Mytilus edulis and Mytilus galloprovincialis. J. Exp. Biol. 205: 1807-1
Mascolo, J. M. & Waite, J. H. (1986) Protein gradients in byssal threads of some marine bivalve molluscs. J. Exp. Zool. 240:1-7
Qin, X. X. & Waite, J. H. (1998) A potential mediator of collagenous block copolymer gradients in mussel byssal threads. Proc. Natl. Acad. Sci. USA 95: 10517-22
Sikorski R. S. & Hieter P. (1989) A system of shuttle vectors and yeast host strains designed for efficient manipulation of DNA in Saccharomyces cerevisiae. Genetics 122, 19-27
Toman, P. D., Chisholm, G., McMullin, H., Giere, L. M., Olsen, D. R., Kovach, R. J., Leigh, S. D., Fong, B. E., Chang, R., Daniels, G. A., Berg, R. A. & Hitzeman, R. A. (2000) Production of recombinant human type I procollagen trimers using a four-gene expression system in the yeast Saccharomyces cerevisiae. J. Biol. Chem. 275: 23303-9
Vaughn, P. R., Galanis, M., Richards, K. M., Tebb, T. A., Ramshaw, J. A. & Werkmeister, J. A. (1998) Production of recombinant hydroxylated human type III collagen fragment in Saccharomyces cerevisiae. DNA Cell Biol. 17: 511-8
Vuorela, A., Myllyharju, J., Nissi, R., Pihlajaniemi, T. & Kivirikko, K. I. (1997) Assembly of human prolyl 4-hydroxylase and type III collagen in the yeast pichia pastoris: formation of a stable enzyme tetramer requires coexpression with collagen and assembly of a stable collagen requires coexpression with prolyl 4-hydroxylase. EMBO J. 16: 6702-12
Waite, J. H., Vaccaro, E., Sun, C. & Lucas, J. M. (2002) Elastomeric gradients: a hedge against stress concentration in marine holdfasts? Philos. Trans. R. Soc. Lond B Biol. Sci. 357: 143-53

1. A yeast cell for producing a recombinant collagen like protein, preferably a recombinant mussel byssus protein, which yeast cell has been transformed with the following elements:
a) a first expression vector which codes for said recombinant collagen like protein; and
b) a second expression vector comprising a nucleic acid coding for prolyl-4-hydroxylase (P4H).

2. The yeast cell of claim 1, wherein the P4H sequence is linked to a signal sequence for efficient transport of said sequence to the ER of said yeast cell.

3. The yeast of claim 2, wherein the signal sequence is mating factor alpha 1 (MFa) of S. cerevisiae (SEQ ID NO: 10).

4. The yeast cell of one or more of claims 1-3, wherein the yeast cell, preferably is a S. cerevisiae, Schizosaccharomyces pombe, Pichia pastoris, Candida albicans, or Hansenula polymorpha cell.

5. The yeast cell of one or more of claims 1-4, wherein the first expression vector further comprises one or more regulatory elements.

6. The yeast cell of claim 5, wherein the regulatory elements contain a promoter selected from constitutive or inducible promoters.

7. The yeast cell of claim 6, wherein the promoter is selected from GPD, GAL4, CUP1, MET25, GAL1 or GAL1-10.

8. The yeast cell of one or more of the preceding claims, wherein the expression vectors are plasmids.

9. The yeast cell of one or more of the preceding claims, wherein the recombinant collagen like protein is a recombinant mussel byssus protein comprising or consisting of one or more fragments of a collagen domain flanked by elastin or silk fibroin.

10. The yeast cell of one or more of the preceding claims, wherein the fragments are derived from Mytilus sp., preferably M. edulis, M. galloprovincialis, M. californians, or Geukeria demissa.

11. The yeast cell of one or more of the preceding claims, wherein the recombinant mussel byssus protein comprises or consists of one or more of the fragments preColP and/or preColD or variants thereof.

12. The yeast cell of one or more of the preceding claims, wherein the recombinant protein comprises or consists of the amino acid sequence of SEQ ID NO: 3 and/or 4 or variants thereof.

13. The yeast cell of one or more of the preceding claims, wherein in the recombinant protein the signal sequence of the respective amino acid sequence is replaced by yeast specific signal sequence, preferably by mating factor alpha 1 (MFa) of S. cerevisiae.

14. The yeast cell of one or more of the preceding claims, wherein P4H is human or mussel P4H.

15. A kit of parts or a co-expression system for use in the production of recombinant collagen comprising proteins comprising the following constituents:
a) the first expression vector as defined in one or more of claims 1-14; and
b) the second expression vector as defined in one or more of claims 1-14;

16. A method of producing a recombinant collagen like protein, preferably mussel byssus protein, comprising the steps of:
a) providing a yeast cell;
b) transforming said yeast cell with a first and second expression vector as defined in one or more of claims 1-14 or with the co-expression system of claim 15;
c) expressing the recombinant collagen like protein, preferably recombinant mussel byssus protein, from said yeast cell under suitable conditions; and
d) recovering said recombinant protein.

17. A method for producing threads from recombinant mussel byssus protein, comprising the following steps:
a) providing recombinant protein as produced in claim 16, and
b) (electro)spinning or molding said protein into threads by a suitable method.

18. A protein obtainable by the method of claim 16 or a thread obtainable by the method of claim 17.

19. Use of the proteins/threads of claim 18 in the field of biotechnology and/or medicine.

20. Use of the proteins/threads of claim 18 for the manufacture of wound closure or coverage systems.

21. The use of claim 20 for the manufacture of suture materials.

22. The use of claim 21, wherein the suture material is intended for use in neurosurgery or ophthalmic surgery.

23. Use of the proteins/threads of claim 18 for the manufacture of replacement materials, preferably artificial cartilage or tendon materials.

24. Wound closure or coverage systems, suture materials, replacement materials, preferably artificial cartilage or tendon materials, which are obtainable using proteins/threads of claim 18.

25. Cosmetics, drug delivery vehicles, fabrics, textile, paper product, leather product, automotive parts or aircraft parts, which contain proteins/threads of claim 18.


SEQUENCE LISTING (10 sequences)

SEQ ID NO: 1 - DNA, 2703 bp, Mytilus edulis
atggttcggttctccctagcatcggtactattactggcagtcaccagcacagctttcgctggaccagttagtgattatggtggtggtggaatcaaagtagtaccctaccacggaggcggaggtggaagcggcggcggtggcggtggaggccatggcggaagcggtattggtggtatcggaggaggatcatcacatgcacatgcccactcttcagcatctgcccatgtgcaccattttggaccaggtggatcttcacatgcatcagctggttcatcatcccatgcatccgcatcccataacggtttaggaggtggcagtgctcatgcacatagcagttccagcgccaacgctcattccggtggattcggtggattcggcggtattggtggtattggcggtattggcccaggaggaagtgtcggaggcggtattggcccaggaggaagtgtcggaggcggcattggcggtattggcggtattggcggcggtggtggaccaggcggtaatggcggtatcggattcggaccaggattcggaggaggattcggaccaggttcatctgctagtggatccggaagtggcagcgcattcggtggtccaggaggttcaagcgcaagcgcaaacgcagctgcacgtgcaaatgcaaatggtggtggaggattcggtggaccaggtaccccaggaaactcaggaccaccaggccaacccggactaccaggagcaccaggccaaccaggacgtccaggaagtaccccaccaggtcgaccaggaaaccccggaccaccaggtcaaccaggtaacccaggacgtccaggctcttcaggaagaccaggaggatccggccaaccaggaggtccaggacgtccaggaacccccggcaaaccaggaaaccgaggacaaccaggacagccaggcggcccaggacaaccaggtcacccaggagcaggaggacaaccaggacgaaacggaaatccaggaaaccccggtaaaccaggaacaccaggtcacccaggaacagcaggatcacgaggaatgccaggaaccccaggaaccccaggacaaccaggaattccaggcaccgtcggaggacgaggaccaagaggaccagctggaatcatcggattaattggaccaaaaggaaatccaggagagccaggaaatccaggtgcaccaggaggcccaggatctacaggaccacaaggaccacaaggaccagccggaggaccaggagcatcaggcggaccaggagacaaaggcgcaccaggtacaccaggaggaactggaccaagaggaccaatcggaccatcaggaccatcaggagcaccaggggaccaaggaccacaaggaggtagaggaacaccaggactcgcaggcaaaccaggacctaaaggactacaaggatcaaatggagaagttggaccccaaggaccatctggacccgcaggaccacaaggcccacaaggaaagaacggtgtcaaaggagcagcaggagatcaaggagctaggggaccagaaggaaaagccggaccagctggaccacaaggagaaacaggaccaaaaggaccaacaggagcacaaggaccagccggtccagccggaccatcaggagaacaaggaccaggaggggaaagaggaggccagggaccacaaggagctgaaggaccaagtggaccagcaggaccaagaggaccagcaggatcacaaggaccaagtggtgaacgcggagaaccaggagcaccaggtaaaaaaggaccaaatggagaccgaggaaaccaaggatcaccaggagcaccaggcaaaaacggagcacgaggaaatagaggatcaagaggaagcaacggatcacccggcagatcaggatcaccaggaagccgaggaaaaccaggaccacaaggaccacatggaccaagaggagcaagaggatcaccaggacaaaaaggaccacgtggagaccaaggagcaccaggtgttattcgtattgttatcgatgaccagagaacaggaccagaagttgcagaattcccaggatttggtggattcggaggagcttcagctaacgcagcaagttcagcaaatgcatttgctggtggacccggtggttccgctggagcaggttcatcatcaggagctaacgcaaacgcaggtggattcccattcggaggaggaccattcggaggagcaggaggtggtcccggagcagcaggaggcccaggaggagcaggaggcccaggaggagtaggaggaggagttggaggtggaccaggaggagtaggaggtggagtaggaggtggaccaggaggagtaggaggtggaccaggaggagcaggaccaggaggagcaggaggatttggaccaggaggagcaggaggatttggtggatttggaggaggatctagcgctggagcatcatcatcaggatcagcatctgcatctaacggtggaccattcggagtactcaatgtaggacccggaggtagaatcggtggtggaagcgcatcagcatctgcagcatctagagcacatgcacacgcttttggtggtctcggagggggaagtgcctcagctggtagtcattcctcatctagctcacactcatttggcggacacgtattccacagtgtgacccatcatggaggtccatcacatgtttcaagcggaggtcacggaggtcatggaggaggtccatacaaacctggatattaa

SEQ ID NO: 2 - DNA, 2025 bp, Mytilus edulis
atggtctacaaactcctgaccgtgtgtcttgtagcatctcttctagagatttgcttagctgactataacggcaacaaacagtatggcggcagatacggcaacagatacggaaacggtttaggaggcggtaatggtggtgcaggagccgtagcccatgcccatgcccatgcccatgccagtgccggagcaaacggaagagcaagagcacatgcacgagccttggcccatgcacatgccggtggtggcgctgcacatggacacccaggattcccagttggtggtagcgcaagcgcagccgcacgagcagcagcacgagcatcagcaggaggattaggtggattcggatcagcagcagccaatgcagcagcagcagcaagagcaggagcaggatttggtggattcggtggattaggaggattcggaggactcggaggagttggcggtccaggtcaaccaggacatgccggtaaacacggaaccgcaggagcagcaggcaaagcaggacgtccaggaccatgtggagatagaggggcaccaggagtaccaggcaaacaaggaccagtaggaggacaaggaccagcaggaccacgaggaccacgaggagatgaaggaccagttggaccaaagggcgaaccaggagcaagaggagctgatggtaaaccaggagacaaaggacctgatggagaaaccggaccacaaggaccagctggaccaaagggacaagtaggagaccaaggcaaaccaggagcaaagggagaaaccggagatcaaggagcacgaggtgaagcaggaaaggccggcgaacaaggaccaggaggcatccaaggaccaaagggaccagtaggaggacaaggaccagcaggaccagccggaccactcggaccacaaggaccaatgggtgaacgaggaccacaaggaccaacaggatcagaaggaccagttggagcaccaggaccaaagggatcagtcggagaccaaggagcacaaggagaccaaggagcaactggcgctgatggcaaaaagggagaaccaggagagagaggacaacaaggagcagcaggaccagtcggccgaccaggaccaagaggagatagaggagcaaagggaattcaaggaagccgaggacgaccaggtggtatgggtagacgaggaaaccgtggatcccaaggagcagtaggaccacgaggagaaactggcccagacggtaaccaaggacaacgtggagaacaaggagcaccaggagttatcacccttgtcattgaagacctcagaacagccggagtagaaagccccgtagaaacctttgacgcaggagcaggaaccggtggaccagcaccaggagtaggagcagcagcaacagcaggagcatttgcaggagcaggaccaggaggagctaatgcaggaggaaacgcagccgcaggagcaggaccaggagtaggaccaggaggactcggaggactaggaggacttggtgcaggtggactcggaggtggactcggcggtggactcggaggattaggaggagcaggaggtttaggtggtggactcggaggattaggaggaggtttaggtggtggactcggaggtttaggaggtggagcaggaggagcaggaggcgcaggagcaggaggaaacggtggagcaggagcaggaggagcaggaggaaacggtggaggatcagccgcagcacgagcagcagcacaagcagcagcagcagcaggaggaaacggtggagcagcacaagcagcagcacaagcagcagcatcagcagcagcaaattcaggacttggagcaggagcagcaagagcagcagcatcagcagccgctagagcaaccgtagcaggacatggaagtggaaccgccgcagcagcagccaacgcagccgcacaagcacatgcagcaacacgaggacaaggaggatcacacgcacacgctgccgccgcagctcacgcagccgcaagtagcgtaatccatggtggtgactatcacggaaacgatgccggctatcacaaaccaggatattaa

SEQ ID NO: 3 - protein, 900 aa, Mytilus edulis
MVRFSLASVLLLAVTSTAFAGPVSDYGGGGIKVVPYHGGGGGSGGGGGGGHGGSGIGGIGGGSSHAHAHSSASAHVHHFGPGGSSHASAGSSSHASASHNGLGGGSAHAHSSSSANAHSGGFGGFGGIGGIGGIGPGGSVGGGIGPGGSVGGGIGGIGGIGGGGGPGGNGGIGFGPGFGGGFGPGSSASGSGSGSAFGGPGGSSASANAAARANANGGGGFGGPGTPGNSGPPGQPGLPGAPGQPGRPGSTPPGRPGNPGPPGQPGNPGRPGSSGRPGGSGQPGGPGRPGTPGKPGNRGQPGQPGGPGQPGHPGAGGQPGRNGNPGNPGKPGTPGHPGTAGSRGMPGTPGTPGQPGIPGTVGGRGPRGPAGIIGLIGPKGNPGEPGNPGAPGGPGSTGPQGPQGPAGGPGASGGPGDKGAPGTPGGTGPRGPIGPSGPSGAPGDQGPQGGRGTPGLAGKPGPKGLQGSNGEVGPQGPSGPAGPQGPQGKNGVKGAAGDQGARGPEGKAGPAGPQGETGPKGPTGAQGPAGPAGPSGEQGPGGERGGQGPQGAEGPSGPAGPRGPAGSQGPSGERGEPGAPGKKGPNGDRGNQGSPGAPGKNGARGNRGSRGSNGSPGRSGSPGSRGKPGPQGPHGPRGARGSPGQKGPRGDQGAPGVIRIVIDDQRTGPEVAEFPGFGGFGGASANAASSANAFAGGPGGSAGAGSSSGANANAGGFPFGGGPFGGAGGGPGAAGGPGGAGGPGGVGGGVGGGPGGVGGGVGGGPGGVGGGPGGAGPGGAGGFGPGGAGGFGGFGGGSSAGASSSGSASASNGGPFGVLNVGPGGRIGGGSASASAASRAHAHAFGGLGGGSASAGSHSSSSSHSFGGHVFHSVTHHGGPSHVSSGGHGGHGGGPYKPGY

SEQ ID NO: 4 - protein, 674 aa, Mytilus edulis
MVYKLLTVCLVASLLEICLADYNGNKQYGGRYGNRYGNGLGGGNGGAGAVAHAHAHAHASAGANGRARAHARALAHAHAGGGAAHGHPGFPVGGSASAAARAAARASAGGLGGFGSAAANAAAAARAGAGFGGFGGLGGFGGLGGVGGPGQPGHAGKHGTAGAAGKAGRPGPCGDRGAPGVPGKQGPVGGQGPAGPRGPRGDEGPVGPKGEPGARGADGKPGDKGPDGETGPQGPAGPKGQVGDQGKPGAKGETGDQGARGEAGKAGEQGPGGIQGPKGPVGGQGPAGPAGPLGPQGPMGERGPQGPTGSEGPVGAPGPKGSVGDQGAQGDQGATGADGKKGEPGERGQQGAAGPVGRPGPRGDRGAKGIQGSRGRPGGMGRRGNRGSQGAVGPRGETGPDGNQGQRGEQGAPGVITLVIEDLRTAGVESPVETFDAGAGTGGPAPGVGAAATAGAFAGAGPGGANAGGNAAAGAGPGVGPGGLGGLGGLGAGGLGGGLGGGLGGLGGAGGLGGGLGGLGGGLGGGLGGLGGGAGGAGGAGAGGNGGAGAGGAGGNGGGSAAARAAAQAAAAAGGNGGAAQAAAQAAASAAANSGLGAGAARAAASAAARATVAGHGSGTAAAAANAAAQAHAATRGQGGSHAHAAAAAHAAASSVIHGGDYHGNDAGYHKPGY

SEQ ID NO: 5 - protein, 536 aa, Saccharomyces cerevisiae
MRFPSIFTAVLFAASSALAHPGFFTSIGQMTDLIHTEKDLVTSLKDYIKAEEDKLEQIKKWAEKLDRLTSTATKDPEGFVGHPVNAFKLMKRLNTEWSELENLVLKDMSDGFISNLTIQRPVLSNDEDQVGAAKALLRLQDTYNLDTDTISKGNLPGVKHKSFLTAEDCFELGKVAYTEADYYHTELWMEQALRQLDEGEISTIDKVSVLDYLSYAVYQQGDLDKALLLTKKLLELDPEHQRANGNLKYFEYIMAKEKDVNKSASDDQSDQKTTPKKKGVAVDYLPERQKYEMLCRGEGIKMTPRRQKKLFCRYHDGNRNPKFILAPAKQEDEWDKPRIIRFHDIISDAEIEIVKDLAKPRLSRATVHDPETGKLTTAQYRVSKSAWLSGYENPVVSRINMRIQDLTGLDVSTAEELQVANYGVGGQYEPHFDFARKDEPDAFKELGTGNRIATWLFYMSDVSAGGATVFPEVGASVWPKKGTAVFWYNLFASGEGDYSTRHAACPVLVGNKWVSNKWLHERGQEFRRPCTLSELE

SEQ ID NO: 6 - protein, 510 aa, Saccharomyces cerevisiae
MRFPSIFTAVLFAASSALADAPEEEDHVLVLRKSNFAEALAAHKYLLVEFYAPWCGHCKALAPEYAKAAGKLKAEGSEIRLAKVDATEESDLAQQYGVRGYPTIKFFRNGDTASPKEYTAGREADDIVNWLKKRTGPAATTLPDGAAAESLVESSEVAVIGFFKDVESDSAKQFLQAAEAIDDIPFGITSNSDVFSKYQLDKDGVVLFKKFDEGRNNFEGEVTKENLLDFIKHNQLPLVIEFTEQTAPKIFGGEIKTHILLFLPKSVSDYDGKLSNFKTAAESFKGKILFIFIDSDHTDNQRILEFFGLKKEECPAVRLITLEEEMTKYKPESEELTAERITEFCHRFLEGKIKPHLMSQELPEDWDKQPVKVLVGKNFEDVAFDEKKNVFVEFYAPWCGHCKQLAPIWDKLGETYKDHENIVIAKMDSTANEVEAVKVHSFPTLKFFPASADRTVIDYNGERTLDGFKKFLESGGQDGAGDDDDLEDLEEAEEPDMEEDDDQKAVKDEL

SEQ ID NO: 7 - DNA, 3876 bp, Mytilus edulis
ccgcggtcattacagttcatctttcacagctttctgatcatcgtcttcctccatgtctggctcctctgcttcttccaggtcctcgagatcgtcatcatcccctgccccatcctggccacccgagaggtccttaaagaattttggtaggtcgcacgcaaggggcaacattagttactggcacctgtcggcactggcaggaaagaacttgagtgtggggaagctgtgcactttgacggcctccacctcgttggcagtcgagtccatcttggcgatgacgatgttctcatggtccttgtacgtctctcccagtttatcccaaatgggagccaactgtttgcagtgaccacaccatggggcatagaactccacaaagacgttttttttctcatcaaaagccacgtcttcaaagttcttcccaacaagcaccttgacaggctgcttgtcccagtcctccggcagctcctggctcatcaggtggggcttgattttgccctccaggaagcggtggcagaactctgtgatcctctctgccgtcagctcctccgattcgggcttgtacttggtcatctcctcctccagggtgatgaggcgcacggccgggcactcttccttcttcaggccaaagaactcgaggatgcgctggttgtcggtgtggtcgctgtcgatgaagatgaacaggatcttgcccttgaagctctcggctgctgttttgaagttgctcagtttgccgtcatagtcagacacactcttgggcaagaacagcaggatgtgagtcttgatttcacctccaaaaatcttcggggctgtctgctcggtgaactcgatgacaaggggcagctggttgtgtttgataaagtccagcaggttctccttggtgacctccccttcaaagttgttccggccttcatcaaacttcttaaagaggacaaccccatctttgtcgagctggtatttggagaacacgtcactgttggaagtgatcccaaatggtatgtcatcgatggcctctgctgcctgcaaaaactgcttggcagagtccgactccacgtccttgaagaagccgatgacagccacctcgctggactccaccaaggactctgcagctgcgccgtcaggcagggtggtggcagccgggcccgtgcgcttcttcagccagttcacgatgtcatcagcctctctgccagctgtatattccttgggggaagccgtgtctccattcctgaagaacttgatggtgggatagccgcgcacgccgtactgctgggccaggtcagactcctccgtggcgtccaccttggccaacctgatctcggaaccttctgccttcagcttcccagcggctttggcatactcaggggccagagccttgcagtggccacaggttccccgtatcttgaggtggtcgtccatgaacacccggcggtcgcggaggcgcttcaagcttttccgcagcaccaggacgtggtcctcctcctccggagcgtcagctaatgcggaggatgctgcgaataaaactgcagtaaaaattgaaggaaatctcatggatccggggttttttctccttgacgttaaagtatagaggtatattaacaattttttgttgatacttttattacatttgaataagaagtaatacaaaccgaaaatgttgaaagtattagttaaagtggttatgcagtttttgcatttatatatctgttaatagatcaaaaatcatcgcttcgctgattaattaccccagaaataaggctaaaaaactaatcgcattatcatcctatggttgttaatttgattcgttcatttgaaggtttgtggggccaggttactgccaatttttcctcttcataaccataaaagctagtattgtagaatctttattgttcggagcagtgcggcgcgaggcacatctgcgtttcaggaacgcgaccggtgaagacgaggacgcacggaggagagtcttccttcggagggctgtcacccgctcggcggcttctaatccgtacttcaatatagcaatgagcagttaagcgtattactgaaagttccaaagagaaggtttttttaggctaagataatggggctctttacatttccacaacatataagtaagattagatatggatatgtatatggatatgtatatggtggtaatgccatgtaatatgattattaaacttctttgcgtccatccaaaaaaaaagtaagaatttttgaaaattcaaggaattcgatatcaagcttatcgataccgtcgacatgagatttccttcaatttttactgcagttttattcgcagcatcctccgcgctagctcatccaggcttttttacttcaattggtcagatgactgatttgatccatactgagaaagatctggtgacttctctgaaagattatattaaggcagaagaggacaagttagaacaaataaaaaaatgggcagagaagttagatcggctaactagtacagcgacaaaagatccagaaggatttgttgggcatccagtaaatgcattcaaattaatgaaacgtctgaatactgagtggagtgagttggagaatctggtccttaaggatatgtcagatggctttatctctaacctaaccattcagagaccagtactttctaatgatgaagatcaggttggggcagccaaagctctgttacgtctccaggatacctacaatttggatacagataccatctcaaagggtaatcttccaggagtgaaacacaaatcttttctaacggctgaggactgctttgagttgggcaaagtggcctatacagaagcagattattaccatacggaactgtggatggaacaagccctaaggcaactggatgaaggcgagatttctaccatagataaagtctctgttctagattatttgagctatgcggtatatcagcagggagacctggataaggcacttttgctcacaaagaagcttcttgaactagatcctgaacatcagagagctaatggtaacttaaaatattttgagtatataatggctaaagaaaaagatgtcaataagtctgcttcagatgaccaatctgatcagaaaactacaccaaagaaaaaaggggttgctgtggattacctgccagagagacagaagtacgaaatgctgtgccgtggggagggtatcaaaatgacccctcggagacagaaaaaactcttttgccgctaccatgatggaaaccgtaatcctaaatttattctggctccagctaaacaggaggatgaatgggacaagcctcgtattattcgcttccatgatattatttctgatgcagaaattgaaatcgtcaaagacctagcaaaaccaaggctgagccgagctacagtacatgaccctgagactggaaaattgaccacagcacagtacagagtatctaagagtgcctggctctctggctatgaaaatcctgtggtgtctcgaattaatatgagaatacaagatctaacaggactagatgtttccacagcagaggaattacaggtagcaaattatggagttggaggacagtatgaaccccattttgactttgcacggaaagatgagccagatgctttcaaagagctggggacaggaaatagaattgctacatggctgttttatatgagtgatgtgtctgcaggaggagccactgtttttcctgaagttggagctagtgtttggcccaaaaaaggaactgctgttttctggtataatctgtttgccagtggagaaggagattatagtacacggcatgcagcctgtccagtgctagttggcaacaaatgggtatccaataaatggctccatgaacgtggacaagaatttcgaagaccttgtacgttgtcagaattggaatgatgagggccc

SEQ ID NO: 8 - protein, 922 aa, Mytilus edulis
  MISC_FEATURE (307)..(307): Xaa can be any naturally occurring amino acid
MVYKLLTVCLVASLLEICLADYNGNKQYGGRYGNRYGNGLGGGNGGAGAVAHAHAHAHASAGANGRARAHARALAHAHAGGGAAHGHPGFPVGGSASAAARAAARASAGGLGGFGSAAANAAAAARAGAGFGGFGGLGGFGGLGGVGGPGQPGGPGGPGGPGGPGGPGMPGGPGGPSGPGTGGPGQPGGPGGPGGPGGPGGPSMPGGPGGPGGPGMPGGPGGPGGPGGAGGIPGMTGPAGPPGPAGPQGPEGEQGPRGRTPAGTPGPPGNPGEPGQGGAPGAPGAPGHAGKHGTAGAAGKAGRPGPXGQAGASGSSGQHGASGAPGRPGNPGSTGRPGATGDPGRPGATGTTGRPGPSGAPGNPGAPGALGAPGPRGSPGFVGLPGPRGSPGEPGNQGPIGGPGYPGPRGPQGPDGAMGPQGPCGDRGAPGVPGKQGPVGGQGPAGPRGPRGDEGPVGPKGEPGARGADGKPGDKGPDGETGPQGPAGPKGQVGDQGKPGAKGETGDQGARGEAGKAGEQGPGGIQGPKGPVGGQGPAGPAGPLGPQGPMGERGPQGPTGSEGPVGAPGPKGSVGDQGAQGDQGATGADGKKGEPGERGQQGAAGPVGRPGPRGDRGAKGIQGSRGRPGGMGRRGNRGSQGAVGPRGETGPDGNQGQRGEQGAPGVITLVIEDLRTAGVESPVETFDAGAGTGGPAPGVGAAATAGAFAGAGPGGANAGGNAAAGAGPGVGPGGLGGLGGLGAGGLGGGLGGGLGGLGGAGGLGGGLGGLGGGLGGGLGGLGGGAGGAGAGGNGGAGAGGAGGNGGGSAAARAAAQAAAAAGGNGGAAQAAAQAAASAAANSGLGAGAARAAASAAARATVTGHGSGTAAAAANAAAQAHAATRGQGGSHAHAAAAAHAAASSVIHGGDYHGNDAGYHKPGY

SEQ ID NO: 9 - protein, 19 aa, Saccharomyces cerevisiae
MRFPSIFTAVLFAASSALA

SEQ ID NO: 10 - protein, 952 aa, Mytilus edulis
MVRFSLASVLLLAVTSTAFAGPVSDYGGGGIKVVPYHGGGGGGGGGGGGGHGGSFRNGRHGGIGGIGGGSSHAHAHSSASAHVHHFGPGGSSHASAGSSSHASASHNGLGGGSAHAHSSSSANAHSGGFGGFGGIGGIGGIGPGGSVGGGIGPGGSVGGGIGGIGGIGGGGGPGGNGGIGFGPGFGGGFGPGSSASGSGSGSAFGGPGGSSASANAAARANANGGGGFGGPGTPGNSGPPGQPGLPGAPGQPGRPGSTPPGRLGNPGPPGQPGNPGRPGSSGRPGGSGQPGGPGRPGTPGKPGNRGQPGQPGGPGQPGHPGAGGQPGRNGNPGNPGKPGTPGHPGTAGSRGMPGTPGTPGQPGIPGTVGGRGPRGPAGIIGLIGPKGNPGEPGNPGAPGGPGSTGPQGPQGPAGGPGASGGPGDKGAPGTPGGTGPRGPIGPSGPSGAPGDQGPQGGRGTPGLAGKPGPKGLQGSNGEVGPQGPSGPAGPQGPQGKNGVKGAAGDQGARGPEGKAGPAGPQGETGPKGPTGAQGPAGPAGPSGEQGPGGERGGQGPQGAEGPSGPAGPRGPAGSQGPSGERGEPGAPGKKGPNGDRGNQGSPGAPGKNGARGNRGSRGSNGSPGRSGSPGSRGKPGPQGPHGPRGLRGSPGQKGPRGDQGAPGVIRIVIDDQRTGPEVAEFPGFGGFGGASANAASSANAFAGGPGGSAGAGSSSGANANAGGFPFGGAGGGPGAAGGPGGAGGPGGVGGGVGGGPGGVGGGVGGGPGGVGGGPGGAGPGGAGGFGPGGAGGFGGFGGGSSAGASSSGSASASNGGPFGVLNVGPGGRIGGGSASASAASRAHAHFGGGSSAGASSSGSASASNGGPFGVLNVGPGGRIGGGSASASAASRAHAHAFGGLGGGSASAGSHSSSSSHSFGGHVFHSVTHHGGPSHVSSGGHGGHGGGPYKPGY